(19) United States
(12) Patent Application Publication
(10) Pub. No.: US 2012/0101086 A1
(43) Pub. Date: Apr. 26, 2012

(54) THIADIAZOLE DERIVATIVES AND THEIR USE FOR THE TREATMENT OF DISORDERS MEDIATED BY S1P1 RECEPTORS

(75) Inventors: James Matthew Bailey, Stevenage (GB); John Alexander Brown, Stevenage (GB); Emmanuel Hubert Demont, Stevenage (GB); Lee Andrew Harrison, Stevenage (GB); Gail Astra Lorraine Seal, Stevenage (GB); Christian Alan Paul Smethurst, Essex (GB); Jason Witherington, Stevenage (GB)

(73) Assignee: GLAXO GROUP LIMITED, Greenford, Middlesex (GB)

(21) Appl. No.: 13/379,110

(22) PCT Filed: Jun. 17, 2010
(86) PCT No.: PCT/EP2010/058514

§ 371 Date: Dec. 19, 2011

(30) Foreign Application Priority Data
Jun. 19, 2009 (GB) 0910688.1

(51) Int. Cl. A61K 31/55; C07D 487/04; C07D 471/04; A61P 25/00; A61P 37/00; A61P 29/00; A61P 11/06; A61P 19/02; A61P 41/00; A61P 1/00; A61P 35/00; A61P 35/04; A61P 7/00; A61P 31/12; A61P 3/10; A61K 31/437

(52) U.S. Cl. 514/215; 514/303; 540/578; 546/119

(57) ABSTRACT

The present invention relates to novel compounds having pharmacological activity, processes for their preparation, pharmaceutical compositions containing them and their use in the treatment of various disorders.

The present invention relates to novel compounds having pharmacological activity, processes for their preparation, pharmaceutical compositions containing them and their use in the treatment of various disorders.

Sphingosine 1-phosphate (S1P) is a bioactive lipid mediator formed by the phosphorylation of sphingosine by sphingosine kinases and is found in high levels in the blood. It is produced and secreted by a number of cell types, including those of hematopoietic origin such as platelets and mast cells (Okamoto et al 1998 J Biol Chem 273(42):27104; Sanchez and Hla 2004, J Cell Biochem 92:913). It has a wide range of biological actions, including regulation of cell proliferation, differentiation, motility, vascularisation, and activation of inflammatory cells and platelets (Pyne and Pyne 2000, Biochem J. 349: 385). Five subtypes of S1P responsive receptor have been described, S1P1 (Edg-1), S1P2 (Edg-5), S1P3 (Edg-3), S1P4 (Edg-6), and S1P5 (Edg-8), forming part of the G-protein coupled endothelial differentiation gene family of receptors (Chun et al 2002 Pharmacological Reviews 54:265, Sanchez and Hla 2004 J Cellular Biochemistry, 92:913). These 5 receptors show differential mRNA expression, with S1P1-3 being widely expressed, S1P4 expressed on lymphoid and hematopoietic tissues and S1P5 primarily in brain and to a lower degree in spleen. They signal via different subsets of G proteins to promote a variety of biological responses (Kluk and Hla 2002 Biochem et Biophysica Acta 1582:72, Sanchez and Hla 2004, J Cellular Biochem 92:913).

Proposed roles for the S1P1 receptor include lymphocyte trafficking, cytokine induction/suppression and effects on endothelial cells (Rosen and Goetzl 2005 Nat Rev Immunol. 5:560). Agonists of the S1P1 receptor have been used in a number of autoimmune and transplantation animal models, including Experimental Autoimmune Encephalomelitis (EAE) models of MS, to reduce the severity of the induced disease (Brinkman et al 2003 JBC 277:21453; Fujino et al 2003 J Pharmacol Exp Ther 305:70; Webb et al 2004 J Neuroimmunol 153:108; Rausch et al 2004 J Magn Reson Imaging 20:16). This activity is reported to be mediated by the effect of S1P1 agonists on lymphocyte circulation through the lymph system. Treatment with S1P1 agonists results in the sequestration of lymphocytes within secondary lymphoid organs such as the lymph nodes, inducing a reversible peripheral lymphopoenia in animal models (Chiba et al 1998, J Immunology 160:5037, Forrest et al 2004 J Pharmacol Exp Ther 309:758; Sanna et al 2004 JBC 279:13839). Published data on agonists suggests that compound treatment induces loss of the S1P1 receptor from the cell surface via internalisation (Graler and Goetzl 2004 FASEB J 18:551; Matloubian et al 2004 Nature 427:355; Jo et al 2005 Chem Biol 12:703) and it is this reduction of S1P1 receptor on immune cells which contributes to the reduction of movement of T cells from the lymph nodes back into the blood stream.

S1P1 gene deletion causes embryonic lethality. Experiments to examine the role of the S1P1 receptor in lymphocyte migration and trafficking have included the adoptive transfer of labelled S1P1 deficient T cells into irradiated wild type mice. These cells showed a reduced egress from secondary lymphoid organs (Matloubian et al 2004 Nature 427:355).

S1P1 has also been ascribed a role in endothelial cell junction modulation (Allende et al 2003 102:3665, Blood Singelton et al 2005 FASEB J 19:1646). With respect to this endothelial action, S1P1 agonists have been reported to have an effect on isolated lymph nodes which may be contributing to a role in modulating immune disorders. S1P1 agonists caused a closing of the endothelial stromal ‘gates’ of lymphatic sinuses which drain the lymph nodes and prevent lymphocyte egress (Wei wt al 2005, Nat. Immunology 6:1228).

The immunosuppressive compound FTY720 (JP11080026-A) has been shown to reduce circulating lymphocytes in animals and man, have disease modulating activity in animal models of immune disorders and reduce remission rates in relapsing remitting Multiple Sclerosis (Brinkman et al 2002 JBC 277:21453, Mandala et al 2002 Science 296:346, Fujino et al 2003 J Pharmacology and Experimental Therapeutics 305:45658, Brinkman et al 2004 American J Transplantation 4:1019, Webb et al 2004 J Neuroimmunology 153:108, Morris et al 2005 EurJ Immunol 35:3570, Chiba 2005 Pharmacology and Therapeutics 108:308, Kahan et al 2003, Transplantation 76:1079, Kappos et al 2006 New Eng J Medicine 335:1124). This compound is a prodrug that is phosphorylated in vivo by sphingosine kinases to give a molecule that has agonist activity at the S1P1, S1P3, S1P4 and S1P5 receptors. Clinical studies have demonstrated that treatment with FTY720 results in bradycardia in the first 24 hours of treatment (Kappos et al 2006 New Eng J Medicine 335:1124). The bradycardia is thought to be due to agonism at the S1P3 receptor, based on a number of cell based and animal experiments. These include the use of S1P3 knock-out animals which, unlike wild type mice, do not demonstrate bradycardia following FTY720 administration and the use of S1P1 selective compounds. (Hale et al 2004 Bioorganic & Medicinal Chemistry Letters 14:3501, Sanna et al 2004 JBC 279:13839, Koyrakh et al 2005 American J Transplantation 5:529)

Hence, there is a need for S1P1 receptor agonist compounds with selectivity over S1P3 which might be expected to show a reduced tendency to induce bradycardia.

The following patent applications describe oxadiazole derivatives as S1P1 agonists: WO03/105771, WO05/058848, WO06/047195, WO06/100633, WO06/115188, WO06/131336, WO07/024,922 and WO07/116,866.

The following patent applications describe tetrahydroisoquinolinyl-oxadiazole derivatives as S1P receptor agonists: WO06/064757, WO06/001463, WO04/113330.

WO08/064,377 describes benzocycloheptyl analogs having S1P1 receptor activity.

A structurally novel class of compounds has now been found which provides agonists of the S1P1 receptor.

The present invention provides compounds of formula (I) or a pharmaceutically acceptable salt thereof:

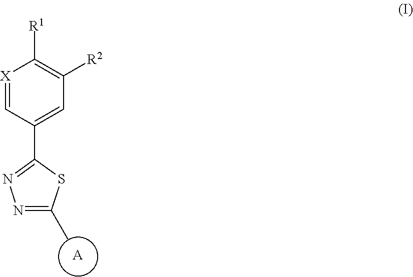

### X is CH or N;

R¹ is OR³, NHR⁴, R⁵, NR⁶R⁷, R⁸ or optionally fluorinated C₍₃₋₆₎cycloalkyl;
R² is hydrogen, halogen, cyano, trifluoromethyl, C₍₁₋₂₎ alkoxy and C₍₁₋₃₎alkyl;
R³ and R⁴ are C₍₁₋₅₎alkyl optionally interrupted by O and optionally substituted by F or (CH₂)₍₀₋₁₎C₍₃₋₅₎cycloalkyl optionally substituted by F;
R⁵ is C₍₁₋₆₎alkyl optionally substituted by F;
R⁶ and R⁷ are independently selected from C₍₁₋₅₎ alkyl optionally interrupted by O and optionally substituted by F and optionally fluorinated C₍₃₋₅₎cycloalkyl with the proviso that the combined number of carbon atoms in R⁶ and R⁷ does not exceed 6;
R⁸ is a 3 to 6 membered, nitrogen-containing heterocyclyl ring optionally substituted by F selected from aziridinyl, azetidinyl, pyrrolidinyl, piperidinyl and morpholinyl, all attached via the nitrogen atom;

A is a bicyclic ring selected from the following:

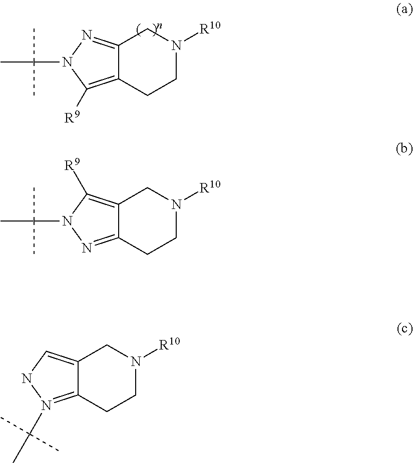

R⁹ is hydrogen or C₍₁₋₃₎alkyl;
R¹⁰ is hydrogen, C₍₁₋₄₎alkyl, C₍₁₋₄₎alkylCOOH, C₍₁₋₄₎alkylCONR¹¹R¹², C₍₂₋₄₎alkylNR¹³CONR¹¹R¹², C₍₂₋₄₎alkylNR¹³COOR¹², C₍₂₋₄₎alkylOCONR¹¹R¹²C₍₂₋₄₎alkylNR¹³COR¹² or COC₍₁₋₄₎NR¹¹R¹²;
when R¹⁰ comprises an alkyl chain of at least two carbon atoms at the point of attachment to the A ring it may be optionally substituted by halogen, SO₂C₍₁₋₃₎alkyl, or by at least one OH;
R¹¹, R¹² and R¹³ are independently selected from hydrogen or C₍₁₋₃₎alkyl optionally substituted by F or hydroxyl and optionally interrupted by O;
R¹¹ and R¹² together with the nitrogen atom to which they are attached may be linked to form a 4-6 membered heterocyclyl ring, wherein the 4- to 6-membered heterocyclyl ring optionally contains an oxygen atom and is optionally substituted by one or two substituents independently selected from F and OH;
R¹² and R¹³, together with the atoms to which they are attached may be linked to form an optionally unsaturated 5-7 membered heterocyclyl ring, wherein the 5- to 7-membered heterocyclyl ring optionally contains an oxygen atom and is optionally substituted by one or two substituents independently selected from F and OH;
n is 1 or 2;
and when R² and R⁹ are C₍₁₋₃₎alkyl, they are optionally substituted by fluorine.

In one embodiment X is CH. In another embodiment X is N.

In one embodiment R¹ is OR³.

In one embodiment R³ is isopropyl.

In one embodiment R² is chloro or cyano.

In one embodiment A is (a) or (b).

In one embodiment R⁹ is hydrogen or methyl.

In one embodiment n is 2.

In one embodiment

### X is CH or N;

### R¹ is OR³;

R³ is isopropyl;
R² is chloro or cyano;

### A is (a) or (b);

R⁹ is hydrogen or methyl; and
n is 2.

The present invention further provides compounds of formula (IA) or a pharmaceutically acceptable salt thereof:

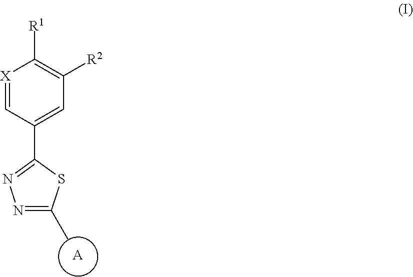

### X is CH or N;

### R¹ is OR³;

R² is halogen or cyano;
R³ is C₍₁₋₅₎ alkyl;
A is a bicyclic ring selected from the following:

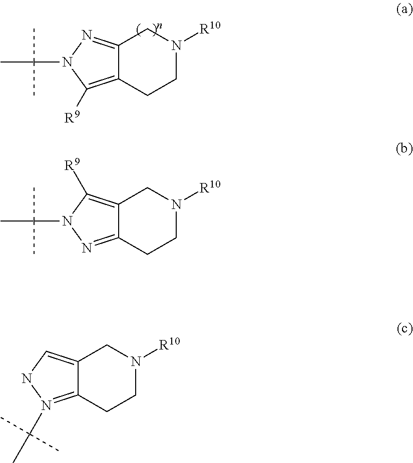

R⁹ is hydrogen or C₍₁₋₃₎alkyl;
R¹⁰ is hydrogen, C₍₁₋₄₎alkyl, C₍₁₋₄₎alkylCOOH, C₍₁₋₄₎alkylCONR¹¹R¹² or COC₍₁₋₄₎NR¹¹R¹²;
when R¹⁰ comprises an alkyl chain of at least two carbon atoms at the point of attachment to the A ring it may be optionally substituted by halogen, SO₂C₍₁₋₃₎alkyl,
OC₍₁₋₃₎alkyl or by at least one OH;
R¹¹, R¹² and R¹³ are independently selected from hydrogen or C₍₁₋₃₎alkyl optionally substituted by F or hydroxyl and optionally interrupted by O; and
n is 1 or 2.

In one embodiment X is CH or N.

In one embodiment R¹ is OR³.

In one embodiment R³ is isopropyl.

In one embodiment R² is chloro or cyano.

In one embodiment A is (a) or (b).

In one embodiment R⁹ is hydrogen or methyl.

In one embodiment R¹⁰ is hydrogen, C₍₃₎alkyl substituted by one or two OH,

C₍₂₎alkylSO₂C₍₁₎alkyl, C₍₁₋₃₎alkylCOOH, C₍₁₋₂₎alkylCON R¹¹R¹², or COC₍₁₋₄₎NR¹¹R¹².

In one embodiment R¹¹ is hydrogen and R¹² is hydrogen, C₍₂₎alkyl substituted by one or two methyl groups and OH or C₍₂₋₃₎alkyl substituted by one or two OH.

In one embodiment n is 1 or 2.

In one embodiment

### X is CH or N;

### R¹ is OR³;

R³ is isopropyl;
R² is chloro or cyano;

### A is (a) or (b);

R² is hydrogen or methyl;
R¹⁰ is hydrogen, C₍₃₎alkyl substituted by one or two OH, C₍₂₎alkylSO₂C₍₁₎alkyl, C₍₁₋₃₎alkylCOOH, C₍₁₋₂₎alkylCON R¹¹R¹², or COC₍₁₋₄₎NR¹¹R¹²;
R¹¹ is hydrogen and R¹² is hydrogen, C₍₂₎alkyl substituted by one or two methyl groups and OH or C₍₂₋₃₎alkyl substituted by one or two OH; and
n is 1 or 2.

The term “alkyl” as a group or part of a group e.g. alkoxy or hydroxyalkyl refers to a straight or branched alkyl group in all isomeric forms. The term “C₍₁₋₆₎alkyl” refers to an alkyl group, as defined above, containing at least 1, and at most 6 carbon atoms Examples of such alkyl groups include methyl, ethyl, propyl, iso-propyl, n-butyl, iso-butyl, sec-butyl, or tert-butyl. Examples of such alkoxy groups include methoxy, ethoxy, propoxy, iso-propoxy, butoxy, iso-butoxy, sec-butoxy and tert-butoxy.

Suitable C₍₃₋₆₎cycloalkyl groups include cyclopropyl, cyclobutyl, cyclopentyl and cyclohexyl.

As used herein, the term “halogen” refers to fluorine (F), chlorine (Cl), bromine (Br), or iodine (I) and the term “halo” refers to the halogen: fluoro (—F), chloro (—Cl), bromo(—Br) and iodo(—I).

The term “substituted” includes the implicit provision that substitution be in accordance with the permitted valence of the substituted atom and the substituent and that the substitution results in a stable compound (i.e. one that does not spontaneously undergo transformation such as by rearrangement, cyclization, or elimination). In certain embodiments, a single atom may be substituted with more than one substituent as long as such substitution is in accordance with the permitted valence of the atom. In certain embodiments, alkyl groups optionally substituted by F or OH may be multiply substituted on multiple carbon atoms.

In certain of the compounds of formula (I), dependent upon the nature of the substituent there are chiral carbon atoms and therefore compounds of formula (I) may exist as stereoisomers. The invention extends to all optical isomers such as stereoisomeric forms of the compounds of formula (I) including enantiomers, diastereoisomers and mixtures thereof, such as racemates. The different stereoisomeric forms may be separated or resolved one from the other by conventional methods or any given isomer may be obtained by conventional stereoselective or asymmetric syntheses.

Certain of the compounds herein can exist in various tautomeric forms and it is to be understood that the invention encompasses all such tautomeric forms.

It is understood that certain compounds of the invention contain both acidic and basic groups and may therefore exist as zwitterions at certain pH values.

Suitable compounds of the invention are:
2-(5-{3-Chloro-4-[(1-methylethyl)oxy]phenyl}-1,3,4-thiadiazol-2-yl)-3-methyl-4,5,6,7-tetrahydro-2H-pyrazolo[4,3-c]pyridine
3-[2-(5-{3-Chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanoic acid
2-[(1-Methylethyl)oxy]-5-[5-(3-methyl-4,5,6,7-tetrahydro-2H-pyrazolo[4,3-c]pyridin-2-yl)-1,3,4-thiadiazol-2-yl]benzonitrile
3-[2-(5-{3-Cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanoic acid
3-[2-(5-{3-Cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanamide
2-(5-{3-Chloro-4-[(1-methylethyl)oxy]phenyl}-1,3,4-thiadiazol-2-yl)-4,5,6,7-tetrahydro-2H-pyrazolo[4,3-c]pyridine
4-[2-(5-{3-Chloro-4-[(1-methylethyl)oxy]phenyl}-1,3,4-thiadiazol-2-yl)-2,4,6,7-tetrahydro-5H-pyrazolo[4,3-c]pyridin-5-yl]butanoic acid
3-[2-(5-{3-Chloro-4-[(1-methylethyl)oxy]phenyl}-1,3,4-thiadiazol-2-yl)-2,4,6,7-tetrahydro-5H-pyrazolo[4,3-c]pyridin-5-yl]propanoic acid
3-[2-(5-{3-Chloro-4-[(1-methylethyl)oxy]phenyl}-1,3,4-thiadiazol-2-yl)-2,4,6,7-tetrahydro-5H-pyrazolo[4,3-c]pyridin-5-yl]-1-propanol
2-(5-{3-Chloro-4-[(1-methylethyl)oxy]phenyl}-1,3,4-thiadiazol-2-yl)-5-[2-(methylsulfonyl)ethyl]-4,5,6,7-tetrahydro-2H-pyrazolo[4,3-c]pyridine
2-[(1-Methylethyl)oxy]-5-[5-(4,5,6,7-tetrahydro-2H-pyrazolo[4,3-c]pyridin-2-yl)-1,3,4-thiadiazol-2-yl]benzonitrile
3-[2-(5-{3-Cyano-4-[(1-methylethyl)oxy]phenyl}-1,3,4-thiadiazol-2-yl)-2,4,6,7-tetrahydro-5H-pyrazolo[4,3-c]pyridin-5-yl]propanoic acid
4-[2-(5-{3-Cyano-4-[(1-methylethyl)oxy]phenyl}-1,3,4-thiadiazol-2-yl)-2,4,6,7-tetrahydro-5H-pyrazolo[4,3-c]pyridin-5-yl]butanoic acid
1-(5-{3-Chloro-4-[(1-methylethyl)oxy]phenyl}-1,3,4-thiadiazol-2-yl)-4,5,6,7-tetrahydro-1H-pyrazolo[4,3-c]pyridine
3-[1-(5-{3-Chloro-4-[(1-methylethyl)oxy]phenyl}-1,3,4-thiadiazol-2-yl)-1,4,6,7-tetrahydro-5H-pyrazolo[4,3-d]pyridin-5-yl]propanoic acid
4-[2-(5-{3-Chloro-4-[(1-methylethyl)oxy]phenyl}-1,3,4-thiadiazol-2-yl)-4,5,7,8-tetrahydropyrazolo[3,4-d]azepin-6(2H)-yl]butanoic acid
2-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-N-[2-hydroxy-1-(hydroxymethyl)ethyl]acetamide
2-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-N-[(1S)-2-hydroxy-1-methylethyl]acetamide
2-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-N-[(1R)-2-hydroxy-1-methylethyl]acetamide
2-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-N-(2-hydroxyethyl)acetamide
2-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-N-[(2S)-2-hydroxypropyl]acetamide
2-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-N-[(2S)-2-hydroxypropyl]acetamide
2-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-N-[(2R)-2-hydroxypropyl]acetamide
5-{5-[5-(2-hydroxyethyl)-3-methyl-4,5,6,7-tetrahydro-2H-pyrazolo[4,3-c]pyridin-2-yl}-1,3,4-thiadiazol-2-yl]-2-[(1-methylethyl)oxy]benzonitrile
5-(5-{5-[2-hydroxy-1-(hydroxymethyl)ethyl]-3-methyl-4,5,6,7-tetrahydro-2H-pyrazolo[4,3-c]pyridin-2-yl}-1,3,4-thiadiazol-2-yl)-2-[(1-methylethyl)oxy]benzonitrile
5-(5-{5-[(2S)-2,3-dihydroxypropyl]-3-methyl-4,5,6,7-tetrahydro-2H-pyrazolo[4,3-c]pyridin-2-yl}-1,3,4-thiadiazol-2-yl)-2-[(1-methylethyl)oxy]benzonitrile
5-(5-{5-[(2R)-2,3-dihydroxypropyl]-3-methyl-4,5,6,7-tetrahydro-2H-pyrazolo[4,3-c]pyridin-2-yl}-1,3,4-thiadiazol-2-yl)-2-[(1-methylethyl)oxy]benzonitrile
5-{5-[5-(3-hydroxypropyl)-3-methyl-4,5,6,7-tetrahydro-2H-pyrazolo[4,3-c]pyridin-2-yl}-1,3,4-thiadiazol-2-yl]-2-[(1-methylethyl)oxy]benzonitrile
5-[5-(5-glycyl-3-methyl-4,5,6,7-tetrahydro-2H-pyrazolo[4,3-c]pyridin-2-yl)-1,3,4-thiadiazol-2-yl]-2-[(1-methylethyl)oxy]benzonitrile
5-[5-(5-{N-[(1R)-2-hydroxy-1-methylethyl]glycyl}-3-methyl-4,5,6,7-tetrahydro-2H-pyrazolo[4,3-c]pyridin-2-yl)-1,3,4-thiadiazol-2-yl]-2-[(1-methylethyl)oxy]benzonitrile
5-[5-(5-{N-[(1S)-2-hydroxy-1-methylethyl]glycyl}-3-methyl-4,5,6,7-tetrahydro-2H-pyrazolo[4,3-c]pyridin-2-yl)-1,3,4-thiadiazol-2-yl]-2-[(1-methylethyl)oxy]benzonitrile
5-[5-(5-{N-[(2R)-2-hydroxypropyl]glycyl}-3-methyl-4,5,6,7-tetrahydro-2H-pyrazolo[4,3-c]pyridin-2-yl)-1,3,4-thiadiazol-2-yl]-2-[(1-methylethyl)oxy]benzonitrile
5-[5-(5-{N-[(2S)-2-hydroxypropyl]glycyl}-3-methyl-4,5,6,7-tetrahydro-2H-pyrazolo[4,3-c]pyridin-2-yl)-1,3,4-thiadiazol-2-yl]-2-[(1-methylethyl)oxy]benzonitrile
5-[5-(5-{N-[(2S)-2,3-dihydroxypropyl]glycyl}-3-methyl-4,5,6,7-tetrahydro-2H-pyrazolo[4,3-c]pyridin-2-yl)-1,3,4-thiadiazol-2-yl]-2-[(1-methylethyl)oxy]benzonitrile
5-(5-{5-[N-(2-hydroxyethyl)glycyl]-3-methyl-4,5,6,7-tetrahydro-2H-pyrazolo[4,3-c]pyridin-2-yl}-1,3,4-thiadiazol-2-yl)-2-[(1-methylethyl)oxy]benzonitrile
5-[5-(5-{N-[2-hydroxy-1-(hydroxymethyl)ethyl]glycyl}-3-methyl-4,5,6,7-tetrahydro-2H-pyrazolo[4,3-c]pyridin-2-yl)-1,3,4-thiadiazol-2-yl]-2-[(1-methylethyl)oxy]benzonitrile
5-[5-(5-{N-[(2R)-2,3-dihydroxypropyl]glycyl}-3-methyl-4,5,6,7-tetrahydro-2H-pyrazolo[4,3-c]pyridin-2-yl)-1,3,4-thiadiazol-2-yl]-2-[(1-methylethyl)oxy]benzonitrile
[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]acetic acid
2-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-1,3-propanediol
(2R)-3-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-1,2-propanediol
methyl (2R)-3-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-2-hydroxypropanoate
(2S)-3-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-1,2-propanediol
2-[(1-methylethyl)oxy]-5-[5-(3-methyl-4,5,6,7-tetrahydro-2H-pyrazolo[3,4-c]pyridin-2-yl)-1,3,4-thiadiazol-2-yl]benzonitrile
2-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-N-(2-hydroxy-1,1-dimethylethyl)acetamide
   or pharmaceutically acceptable salts or esters thereof.

Suitably a compound of formula (I) is
3-[2-(5-{3-Chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanoic acid or a salt or ester thereof.

Suitably a compound of formula (I) is
2-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-1,3-propanediol or a salt or ester thereof.

Pharmaceutically acceptable derivatives of compounds of formula (I) include any pharmaceutically acceptable salt, ester or salt of such ester of a compound of formula (I) which, upon administration to the recipient is capable of providing (directly or indirectly) a compound of formula (I) or an active metabolite or residue thereof.

The compounds of formula (I) can form salts. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. Certain of the compounds of formula (I) may form acid addition salts with one or more equivalents of the acid. The present invention includes within its scope all possible stoichiometric and non-stoichiometric forms. Salts may also be prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropylamine, tris(hydroxymethyl)aminomethane (TRIS, trometamol) and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

Pharmaceutically acceptable acid addition salts may be prepared conventionally by reaction with the appropriate acid or acid derivative. Pharmaceutically acceptable salts with bases may be prepared conventionally by reaction with the appropriate inorganic or organic base.

The compounds of formula (I) may be prepared in crystalline or non-crystalline form, and, if crystalline, may optionally be hydrated or solvated. This invention includes within its scope stoichiometric hydrates or solvates as well as compounds containing variable amounts of water and/or solvent.

Included within the scope of the invention are all salts, solvates, hydrates, complexes, polymorphs, prodrugs, radiolabelled derivatives, stereoisomers and optical isomers of the compounds of formula (I).

The potencies and efficacies of the compounds of this invention for the S1P1 receptor can be determined by GTPγS assay performed on the human cloned receptor as described herein. Compounds of formula (I) have demonstrated agonist activity at the S1P1 receptor, using functional assays described herein.

Compounds of formula (I) and their pharmaceutically acceptable salts are therefore of use in the treatment of conditions or disorders which are mediated via the S1P1 receptor. In particular the compounds of formula (I) and their pharmaceutically acceptable salts are of use in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

Compounds of formula (I) and their pharmaceutically acceptable salts are therefore of use in the treatment of lupus erythematosis.

Compounds of formula (I) and their pharmaceutically acceptable salts are therefore of use in the treatment of psoriasis.

Compounds of formula (I) and their pharmaceutically acceptable salts are therefore of use in the treatment of multiple sclerosis.

Compounds of formula (I) and their pharmaceutically acceptable salts may also be of use in the treatment of Parkinson's Disease, Alzheimer's disease, Huntington's chorea, amyotrophic lateral sclerosis, spinal muscular atrophy, polyglutamine expansion disorders, vascular dementia, Down's syndrome, HIV dementia, dementia, ocular diseases including glaucoma, aged related macular degeneration, cataracts, traumatic eye injury, diabetic retinopathy, traumatic brain injury, stroke, tauopathies and hearing loss.

It is to be understood that “treatment” as used herein includes prophylaxis as well as alleviation of established symptoms.

Thus the invention also provides compounds of formula (I) or pharmaceutically acceptable salts thereof, for use as therapeutic substances, in particular in the treatment of the conditions or disorders mediated via the S1P1 receptor. In particular the invention provides a compound of formula (I) or a pharmaceutically acceptable salt thereof for use as a therapeutic substance in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

Compounds of formula (I) and their pharmaceutically acceptable salts are of use as therapeutic substances in the treatment of lupus erythematosis.

Compounds of formula (I) and their pharmaceutically acceptable salts are of use as therapeutic substances in the treatment of psoriasis.

Compounds of formula (I) and their pharmaceutically acceptable salts are of use as therapeutic substances in the treatment of multiple sclerosis.

The invention further provides a method of treatment of conditions or disorders in mammals including humans which can be mediated via the S1P1 receptor, which comprises administering to the sufferer a therapeutically safe and effective amount of a compound of formula (I) or a pharmaceutically acceptable salt thereof. In particular the invention provides a method of treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes, which comprises administering to the sufferer a therapeutically safe and effective amount of a compound of formula (I) or a pharmaceutically acceptable salt thereof.

The invention provides a method of treatment of lupus erythematosis, which comprises administering to the sufferer a therapeutically safe and effective amount of a compound of formula (I) or a pharmaceutically acceptable salt thereof.

The invention provides a method of treatment of psoriasis, which comprises administering to the sufferer a therapeutically safe and effective amount of a compound of formula (I) or a pharmaceutically acceptable salt thereof.

The invention provides a method of treatment of multiple sclerosis, which comprises administering to the sufferer a therapeutically safe and effective amount of a compound of formula (I) or a pharmaceutically acceptable salt thereof.

In another aspect, the invention provides for the use of a compound of formula (I) or a pharmaceutically acceptable salt thereof in the manufacture of a medicament for use in the treatment of the conditions or disorders mediated via the S1P1 receptor.

In particular the invention provides a compound of formula (I) or a pharmaceutically acceptable salt thereof for use in the manufacture of a medicament for use in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

Compounds of formula (I) and their pharmaceutically acceptable salts are of use in the manufacture of a medicament for use in the treatment of lupus erythematosis.

Compounds of formula (I) and their pharmaceutically acceptable salts are of use in the manufacture of a medicament for use in the treatment of psoriasis.

Compounds of formula (I) and their pharmaceutically acceptable salts are of use in the manufacture of a medicament for use in the treatment of multiple sclerosis.

In order to use the compounds of formula (I) and pharmaceutically acceptable salts thereof in therapy, they will normally be formulated into a pharmaceutical composition in accordance with standard pharmaceutical practice. The present invention also provides a pharmaceutical composition, which comprises a compound of formula (I) or a pharmaceutically acceptable salt thereof, and a pharmaceutically acceptable carrier or excipient.

In a further aspect, the present invention provides a process for preparing a pharmaceutical composition, the process comprising mixing a compound of formula (I) or a pharmaceutically acceptable salt thereof and a pharmaceutically acceptable carrier or excipient.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents (e.g. pregelatinised maize starch, polyvinylpyrrolidone or hydroxypropyl methylcellulose); fillers (e.g. lactose, microcrystalline cellulose or calcium hydrogen phosphate); tabletting lubricants (e.g. magnesium stearate, talc or silica); disintegrants (e.g. potato starch or sodium starch glycollate); and acceptable wetting agents (e.g. sodium lauryl sulphate). The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for oral administration may be suitably formulated to give controlled release of the active compound.

For parenteral administration, fluid unit dosage forms are prepared utilising a compound of the invention or pharmaceutically acceptable salts thereof and a sterile vehicle. Formulations for injection may be presented in unit dosage form e.g. in ampoules or in multi-dose, utilising a compound of the invention or pharmaceutically acceptable derivatives thereof and a sterile vehicle, optionally with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of formula (I) or pharmaceutically acceptable salts thereof may also be formulated in rectal compositions such as suppositories or retention enemas, e.g. containing conventional suppository bases such as cocoa butter or other glycerides.

The compounds of formula (I) or pharmaceutically acceptable salts thereof may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of formula (I) or pharmaceutically acceptable salts thereof, may be formulated as solutions for administration via a suitable metered or unitary dose device or alternatively as a powder mix with a suitable carrier for administration using a suitable delivery device. Thus compounds of formula (I) or pharmaceutically acceptable salts thereof may be formulated for oral, buccal, parenteral, topical (including ophthalmic and nasal), depot or rectal administration or in a form suitable for administration by inhalation or insufflation (either through the mouth or nose).

The compounds of formula (I) or pharmaceutically acceptable salts thereof may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

The composition may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, 1.0 to 500 mg or 1.0 to 200 mg and such unit doses may be administered more than once a day, for example two or three times a day.

Compounds of formula (I) or pharmaceutically acceptable salts thereof may be used in combination preparations, in combination with other active ingredients. For example, the compounds of the invention may be used in combination with cyclosporin A, methotrexate, steriods, rapamycin, proinflammatory cytokine inhibitors, immunomodulators including biologicals or other therapeutically active compounds.

The subject invention also includes isotopically-labeled compounds, which are identical to those recited in formulas I and following, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹⁸F, ¹²³I and ¹²⁵I.

Compounds of the present invention and pharmaceutically acceptable salts of said compounds that contain the aforementioned isotopes and/or other isotopes of other atoms are within the scope of the present invention. Isotopically-labeled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., 2H, can afford certain therapeutic advantages resulting from greater metabolic stability, for example increased in vivo half-life or reduced dosage requirements and, hence, may be preferred in some circumstances. Isotopically labelled compounds of formula (I) and following of this invention can generally be prepared by carrying out the procedures disclosed in the Schemes and/or in the Examples below, by substituting a readily available isotopically labelled reagent for a non-isotopically labeled reagent.

In a further aspect, this invention provides processes for preparation of a compound of formula (I).

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of compounds of the invention.

### ABBREVIATIONS

g—grams
mg—milligrams
ml—millilitres
ul—microlitres
BOC₂O—bis(1,1-dimethylethyl) dicarbonate
MeCN—acetonitrile
MeOH—methanol
EtOH—ethanol
Et2O—diethyl ether
EtOAc—ethyl acetate
DBU—1,8-Diazabicyclo[5.4.0]undec-7-ene
DCM—dichloromethane
DIAD—diisopropyl azodicarboxylate
DIPEA—diisopropylethylamine
DME—1,2-bis(methyloxy)ethane
DMF—N,N-dimethylformamide
DMSO—dimethylsulphoxide
d₆DMSO—deuterated dimethylsulphoxide
EDAC—N-(3-Dimethylaminopropyl)-N′-ethylcarbodiimide hydrochloride
EDC—N-(3-Dimethylaminopropyl)-N′-ethylcarbodiimide hydrochloride

EDCI—N-(3-Dimethylaminopropyl)-N′-ethylcarbodiimide hydrochloride

HATU—2-(1H-7-Azabenzotriazol-1-yl)-1,1,3,3-tetramethyl uronium
hexafluorophosphate Methanaminium
HOBT/HOBt—Hydroxybenzotriazole
IPA—isopropylalcohol
MeOD—deuterated methanol
NCS—N-chlorosuccinimide
PPh₃—Triphenylphosphine
PyBOP—Benzotriazol-1-yl-oxytripyrrolidinophosphonium hexafluorophosphate
THF—tetrahydrofuran
TFA—trifluoroacetic acid
dba—dibenzylidene acetone
RT—room temperature
° C.—degrees Celsius
M—Molar
H—proton
s—singlet
d—doublet
t—triplet
q—quartet
MHz—megahertz
MeOD—deuterated methanol

LCMS—Liquid Chromatography Mass Spectrometry
LC/MS—Liquid Chromatography Mass Spectrometry
MS—mass spectrometry
ES—Electrospray
MH+—mass ion+H+
MDAP—mass directed automated preparative liquid chromatography.
sat. —saturated
SCX—solid phase cation exchange chromatography

### LCMS Methodology

LCMS data generated by method formate unless otherwise stated.

### Method Formate

LC conditions

The HPLC analysis was conducted on an Acquity HPLC BEH C18 column (50 mm×2.1 mm, i.d. 1.7 μm packing diameter) at 40° C.

The solvents employed were:
A=0.1% v/v solution of formic acid in water
B=0.1% v/v solution of formic acid in acetonitrile

The gradient employed was:

| Time (min) | Flow rate (ml/min) | % A | % B |
|---|---|---|---|
| 0 | 1 | 99 | 1 |
| 1.5 | 1 | 3 | 97 |
| 1.9 | 1 | 3 | 97 |
| 2.0 | 1 | 0 | 100 |

The UV detection was a summed signal from wavelength of 210 nm to 350 nm.

### MS Conditions

| | |
|---|---|
| MS | Waters ZQ |
| Ionisation mode | Alternate-scan positive and negative electrospray |
| Scan range | 100 to 1000 AMU |
| Scan time | 0.27 sec |
| Inter scan delay | 0.10 sec |

Method Formate 5 min

LC Conditions

The HPLC analysis was conducted on a Sunfire C18 column (30 mm×4.6 mm, i.d. 3.5 μm packing diameter) at 30° C.

The solvents employed were:
A=0.1% v/v solution of formic acid in water
B=0.1% v/v solution of formic acid in acetonitrile

The gradient employed was:

| Time (min) | Flow rate (ml/min) | % A | % B |
|---|---|---|---|
| 0 | 3 | 97 | 3 |
| 0.1 | 3 | 97 | 3 |
| 4.2 | 3 | 0 | 100 |
| 4.8 | 3 | 0 | 100 |
| 4.9 | 3 | 97 | 3 |
| 5.0 | 3 | 97 | 3 |

The UV detection was a summed signal from wavelength of 210 nm to 350 nm.

MS Conditions

| | |
|---|---|
| MS | Waters ZQ |
| Ionisation mode | Alternate-scan positive and negative electrospray |
| Scan range | 100 to 1000 AMU |
| Scan time | 0.50 sec |
| Inter scan delay | 0.20 sec |

### Method HpH

### LC Conditions

The HPLC analysis was conducted on an Acquity HPLC BEH C18 column (50 mm×2.1 mm, i.d. 1.7 μm packing diameter) at 40° C.

The solvents employed were:
A=10 mM ammonium hydrogen carbonate in water adjusted to pH10 with ammonia solution
B=acetonitrile

The gradient employed was:

| Time (min) | Flow rate (ml/min) | % A | % B |
|---|---|---|---|
| 0 | 1 | 99 | 1 |
| 1.5 | 1 | 3 | 97 |
| 1.9 | 1 | 3 | 97 |
| 2.0 | 1 | 0 | 100 |

The UV detection was a summed signal from wavelength of 210 nm to 350 nm.

### MS Conditions

| | |
|---|---|
| MS | Waters ZQ |
| Ionisation mode | Alternate-scan positive and negative electrospray |
| Scan range | 100 to 1000 AMU |
| Scan time | 0.27 sec |
| Inter scan delay | 0.10 sec |

### MDAP Methodology

### Method Formate

LC Conditions

The HPLC analysis was conducted on either a Sunfire C18 column (100 mm×19 mm, i.d 5 μm packing diameter) or a Sunfire C18 column (150 mm×30 mm, i.d. 5 μm packing diameter) at ambient temperature.

The solvents employed were:
A=0.1% v/v solution of formic acid in water
B=0.1% v/v solution of formic acid in acetonitrile

Run as a gradient over either 15 or 25 min (extended run) with a flow rate of 20 ml/min (100 mm×19 mm, i.d 5 μm packing diameter) or 40 ml/min (150 mm×30 mm, i.d. 5 μm packing diameter).

The UV detection was a summed signal from wavelength of 210 nm to 350 nm.

MS conditions

| | |
|---|---|
| MS | Waters ZQ |
| Ionisation mode | Alternate-scan positive and negative electrospray |
| Scan range | 100 to 1000 AMU |
| Scan time | 0.50 sec |
| Inter scan delay | 0.20 sec |

### Method HpH

LC conditions

The HPLC analysis was conducted on either an Xbridge C18 column (100 mm×19 mm, i.d 5 μm packing diameter) or a Xbridge C18 column (100 mm×30 mm, i.d. 5 μm packing diameter) at ambient temperature.

The solvents employed were:
A=10 mM ammonium bicarbonate in water, adjusted to pH10 with ammonia solution
B=acetonitrile

Run as a gradient over either 15 or 25 min (extended run) with a flow rate of 20 ml/min (100 mm×19 mm, i.d 5 μm packing diameter) or 40 ml/min (100 mm×30 mm, i.d 5 μm packing diameter).

The UV detection was a summed signal from wavelength of 210 nm to 350 nm.

### MS Conditions

| | |
|---|---|
| MS | Waters ZQ |
| Ionisation mode | Alternate-scan positive and negative electrospray |
| Scan range | 100 to 1000 AMU |
| Scan time | 0.50 sec |
| Inter scan delay | 0.20 sec |

### General Chemistry Section

The methods described below are given for illustrative purposes. Intermediates in the preparation of the examples may not necessarily have been prepared from the specific batches described.

### PREPARATION 1

### 5-{3-Chloro-4-[(1-methylethyl)oxy]phenyl}-1,3,4-thiadiazol-2-amine

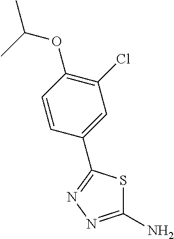

A flask was charged with 3-chloro-4-[(1-methylethyl)oxy]benzoic acid (CAS#: 213598-07-3, commercially available from Boaopharma, 25 g, 116 mmol) and hydrazinecarbothioamide (CAS#: 79-19-6, commercially available from Aldrich, 15.92 g, 175 mmol). Phosphorus oxychloride (CAS#: 10025-87-3, commercially available from Aldrich, 50 ml, 556 mmol) was then cautiously added and the resulting mixture was stirred for 20 minutes at room temperature, at 90° C. for 18 hours. The mixture was very cautiously added dropwise to a vigorously stirred mixture of ice and water (1 l). The resulting mixture was basified (pH 12) with a 10M NaOH aqueous solution and stirred for 30 minutes while cooling with an ice/water bath. The oily sludge remaining was collected by filtration then dissolved in DCM (1 l). The organic phase was washed with brine, dried and concentrated in vacuo to give 5-{3-chloro-4-[(1-methylethyl)oxy]phenyl}-1,3,4-thiadiazol-2-amine (9.8 g, 31.2% yield) as a brown solid which was used in the next step (Preparation 2) without further purification. LCMS: Retention time 1.02 min; [M+H]⁺=270.05

### PREPARATION 1 ALTERNATIVE PROCEDURE

### 5-{3-chloro-4-[(1-methylethyl)oxy]phenyl}-1,3,4-thiadiazol-2-amine

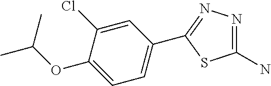

Phosphoric trichloride (17.41 g, 114 mmol) was added to a mixture of hydrazinecarbothioamide (5.17 g, 56.8 mmol) and 3-chloro-4-[(1-methylethyl)oxy]benzoyl chloride (14 g, 37.8 mmol) and the mixture was heated at 90° C. for 3 h. A sample was taken and quenched into a mixture of ice and 10M NaOH, then extracted with EtOAc. The heat was switched off and the mixture allowed to stand at room temperature overnight and then added to cold 5M NaOH solution, cooling in ice bath. The mixture was extracted with EtOAc (2×200 mL), the solvent dried and evaporated to give a beige solid. Product was heated in ethanol (120 mL) until it all dissolved, then cooled in an ice bath and the precipitated solid collected by filtration and dried in vacuo to give 5-{3-chloro-4-[(1-methylethyl)oxy]phenyl}-1,3,4-thiadiazol-2-amine (4.65 g, 45.6%)

1H NMR (DMSO-d6,400 MHz): δ(ppm) 7.79 (d, J=2.0 Hz, 1H), 7.63 (dd, J=8.7, 1.9 Hz, 1H), 7.40 (s, 2H), 7.25 (d, J=8.6 Hz, 1H), 4.75 (spt, J=5.9 Hz, 1H), 1.32 (d, J=5.8 Hz, 6H)

### PREPARATION 2

### 2-Bromo-5-{3-chloro-4-[(1-methylethyl)oxy]phenyl}-1,3,4-thiadiazole

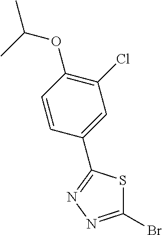

A flask was charged with cupric bromide (15.73 g, 70.4 mmol) and t-butyl nitrite (7.26 g, 70.4 mmol) then filled with CH₃CN (300 ml). The resulting mixture was stirred at room temperature for 30 minutes, then treated with small portions of a slurry of 5-{3-chloro-4-[(1-methylethyl)oxy]phenyl}-1,3,4-thiadiazol-2-amine (Preparation 1) (9.5 g, 35.2 mmol) over 1 hour. The resulting mixture was stirred at room temperature for 1 hour, then at 60° C. for 1 hour then cooled to room temperature and concentrated in vacuo. The residue was dissolved in AcOEt (400 ml) and water (50 ml) added, giving a thick suspension which was filtered through celite. The filtrate was washed with water (400 ml) then brine (300 ml), dried and concentrated in vacuo. Purification of the residue by flash chromatography on silica gel (c-Hexane/AcOEt: 0 to 30% gradient) gave 2-bromo-5-{3-chloro-4-[(1-methylethyl)oxy]phenyl}-1,3,4-thiadiazole (4.4 g, 37%) as a yellow solid.

LCMS: Retention time 1.33 min; [M+H]⁺=333, 335

### PREPARATION 2 ALTERNATIVE PROCEDURE

### 2-Bromo-5-{3-chloro-4-[(1-methylethyl)oxy]phenyl}-1,3,4-thiadiazole

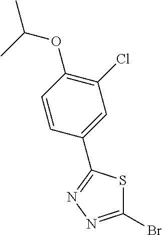

Cupric bromide (8.20 g, 36.7 mmol) and 1,1-dimethylethyl nitrite (4.36 mL, 36.7 mmol) were dissolved in acetonitrile and the mixture was stirred for 10 min, then 5-{3-chloro-4-[(1-methylethyl)oxy]phenyl}-1,3,4-thiadiazol-2-amine (Preparation 1) (4.5 g, 16.68 mmol) was added in small portions over 30 min. The dark brown mixture was stirred at room temperature for 1 h. The mixture was evaporated in vacuo to give a black residue. This was triturated with EtOAc (150 mL), filtered through a thin Celite pad and the pad washed with EtOAc (100 mL), the combined solvent washed with 2M HCl (100 mL) and brine (100 mL), dried and evaporated to give 2-bromo-5-{3-chloro-4-[(1-methylethyl)oxy]phenyl}-1,3,4-thiadiazole as a brown solid (5.42 g, 97%)

LCMS: Retention time 1.32 min; [M+H]⁺=335, 337

1H NMR(CHLOROFORM-d, 400 MHz): δ (ppm) 7.94 (d, J=2.0 Hz, 1H), 7.76 (dd, J=8.6, 2.3 Hz, 1H), 7.02 (d, J=8.8 Hz, 1H), 4.69 (spt, J=6.0 Hz, 1H), 1.44 (d, J=6.1 Hz, 6H)

### PREPARATION 3

### 5-{3-Chloro-4-[(1-methylethyl)oxy]phenyl}-1,3,4-thiadiazol-2(3H)-one hydrazone

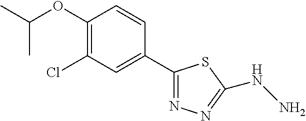

Hydrazine hydrate (1.590 ml, 51.0 mmol) was added to a mixture of 2-bromo-5-{3-chloro-4-[(1-methylethyl)oxy]phenyl}-1,3,4-thiadiazole (Preparation 2) (1.703 g, 5.10 mmol) in i-PrOH (20 ml) and the resulting mixture was stirred at 105° C. under nitrogen for 24 hours then cooled to room temperature and concentrated in vacuo. Purification of the residue by flash chromatography on silica gel (40 g, DCM/MeOH: 0 to 5% gradient) gave 5-{3-chloro-4-[(1-methylethyl)oxy]phenyl}-1,3,4-thiadiazol-2(3H)-one hydrazone (617 mg, 42%) as a cream yellow solid.

LCMS: Retention time 0.97 min; [M+H]⁺=285, 287

### PREPARATION 3 ALTERNATIVE PROCEDURE

### 5-{3-chloro-4-[(1-methylethyl)oxy]phenyl}-1,3,4-thiadiazol-2(3H)-one hydrazone

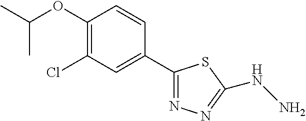

2-bromo-5-{3-chloro-4-[(1-methylethyl)oxy]phenyl}-1,3,4-thiadiazole (33 g, 99 mmol) was suspended in Isopropanol (300 mL) under nitrogen. Hydrazine hydrate (31.0 mL, 989 mmol) was added and the mixture heated to 105° C. overnight. Water (100 mL) was added, the solvent was evaporated in vacuo to half volume, and the solid product collected by filtration to give 5-{3-chloro-4-[(1-methylethyl)oxy]phenyl}-1,3,4-thiadiazol-2(3H)-one hydrazone as orange solid (23.6 g, 84%)

LCMS: Retention time 0.95 min; [M+H]⁺=285, 287

1H NMR (DMSO-d6,400 MHz): δ (ppm) 7.79 (d, J=2.0 Hz, 1H), 7.63 (dd, J=8.7, 1.9 Hz, 1H), 7.40 (s, 2H), 7.25 (d, J=8.6 Hz, 1H), 4.75 (spt, J=5.9 Hz, 1H), 1.32 (d, J=5.8 Hz, 6H)

### PREPARATION 4

### 1,1-Dimethylethyl 3-acetyl-4-oxo-1-piperidinecarboxylate

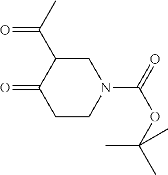

1,1-Dimethylethyl 4-oxo-1-piperidinecarboxylate (CAS#: 79099-07-3, commercially available from Aldrich, 5 g, 25.09 mmol) and pyrrolidine (4.15 ml, 50.2 mmol) were dissolved in toluene (30 ml) and the resulting mixture was refluxed for 3 hours under nitrogen using a Dean and Stark apparatus, then cooled to room temperature and concentrated in vacuo. The residue was dissolved in 1,4-dioxane (25 ml), then acetic anhydride (5.21 ml, 55.2 mmol) was added and the resulting mixture allowed to stand at room temperature under nitrogen overnight. Water (6 ml, 333 mmol) was added and the resulting mixture was refluxed for 1 hour then cooled to room temperature and concentrated in vacuo. The residue was dissolved in water (20 ml) and the aqueous phase was extracted twice with AcOEt (20 ml). The combined organics extracts were washed with a 5% w/w HCl aqueous solution (20 ml), dried over MgSO₄ and concentrated in vacuo to give 1,1-dimethylethyl 3-acetyl-4-oxo-1-piperidinecarboxylate (5.3 g, 88%) as a yellow oil which was used in the next step without further purification.

### PREPARATION 4 ALTERNATIVE PROCEDURE

### 1,1-dimethylethyl 3-acetyl-4-oxo-1-piperidinecarboxylate

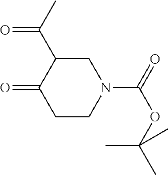

1,1-dimethylethyl 4-oxo-1-piperidinecarboxylate (23.6 g, 118 mmol) and pyrrolidine (19.59 mL, 237 mmol) were dissolved in toluene (30 mL) and the reaction mixture heated at 130° C. under N₂ using a Dean Stark trap to remove water. After 5 h the reaction was allowed to cool to RT, and the solvent evaporated to give a yellow oil. This was dissolved in 1,4-Dioxane (100 mL), then acetic anhydride (24.6 mL, 261 mmol) was added and the reaction allowed to sit at room temperature under N₂ overnight. Water (30.0 mL, 1665 mmol) was added to the orange red solution and the mixture heated at reflux under N2 for 3 h then the reaction allowed to cool to room temperature. The mixture was evaporated to about 50% volume and this solution diluted with EtOAc and washed with water. The organic phase was washed with 5% HCl (20 mL) and then dried over magnesium sulphate and evaporated to give 1,1-dimethylethyl 3-acetyl-4-oxo-1-piperidinecarboxylate (23.5 g, 82% yield) as a yellow oil which was used without purification

LCMS: Retention time 1.02 min; [M−H]⁻=240

1H NMR(CHLOROFORM-d, 400 MHz): δ (ppm) 15.67 (s, 1H), 4.19 (br. s., 2H), 3.59 (t, J=5.8 Hz, 2H), 2.45 (t, J=5.8 Hz, 3H), 2.14 (s, 3H), 1.49 (s, 9H)

### PREPARATION 5

### 1,1-Dimethylethyl 2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridine-5-carboxylate

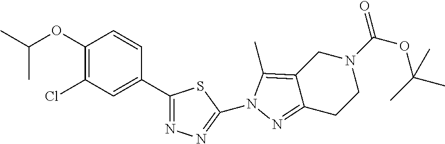

5-{3-Chloro-4-[(1-methylethyl)oxy]phenyl}-1,3,4-thiadiazol-2(3H)-one hydrazone (Preparation 3) (260 mg, 0.912 mmol) and 1,1-dimethylethyl 3-acetyl-4-oxo-1-piperidinecarboxylate (Preparation 4) (200 mg, 0.829 mmol) were dissolved in N,N-dimethylacetamide (5 ml) and the resulting mixture was stirred at 150° C. for 1 hour under microwave irradiation then cooled to room temperature and concentrated in vacuo. Purification of the residue by flash chromatography on silica gel (c-Hexane/AcOEt: 5 to 50% gradient) gave 1,1-dimethylethyl 2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridine-5-carboxylate (245 mg, 60%) as a yellow solid.

LCMS: Retention time 1.58 min; [M+H]⁺=489.9, 491.9

### PREPARATION 5 ALTERNATIVE PROCEDURE

### 1,1-Dimethylethyl 2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridine-5-carboxylate

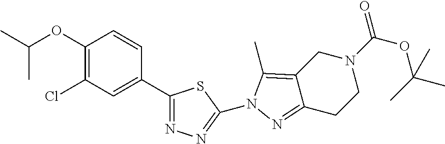

1,1-dimethylethyl 3-acetyl-4-oxo-1-piperidinecarboxylate (Preparation 4) (19.57 g, 81 mmol) and 5-{3-chloro-4-[(1-methylethyl)oxy]phenyl}-1,3,4-thiadiazol-2(3H)-one hydrazone (Preparation 3)(23.1 g, 81 mmol) were suspended in ethanol (300 mL) and acetic acid (0.5 mL) was added, then the suspension heated to reflux for 3 h. The mixture was allowed to cool to room temperature over 40 min, then filtered and the solid washed with ethanol to give 1,1-Dimethylethyl 2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridine-5-carboxylate (19.2 g, 48.3%)

LCMS: Retention time 1.57 min; [M+H]⁺=490, 492

1H NMR(CHLOROFORM-d, 400 MHz): δ (ppm) 7.97 (d, J=2.0 Hz, 1H), 7.76 (dd, J=8.6, 2.0 Hz, 1H), 7.03 (d, J=8.8 Hz, 1H), 4.68 (spt, J=6.1 Hz, 1H), 4.35-4.54 (m, 2H), 3.60-3.88 (m, 2H), 2.81 (br. s., 2H), 2.70 (s, 3H), 1.51 (s, 9H), 1.44 (d, J=6.1 Hz, 6H)

### PREPARATION 6

### 1,1-Dimethylethyl 3-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanoate

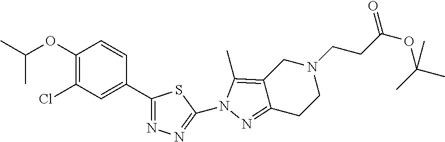

DBU (0.085 ml, 0.562 mmol) was added to a solution of 2-(5-{3-chloro-4-[(1-methylethyl)oxy]phenyl}-1,3,4-thiadiazol-2-yl)-3-methyl-4,5,6,7-tetrahydro-2H-pyrazolo[4,3-c]pyridine (Example 1) (73 mg, 0.187 mmol) and 1,1-dimethylethyl 2-propenoate (0.136 ml, 0.936 mmol) in DMF (5 ml) at room temperature under nitrogen. The resulting mixture was stirred at this temperature for 21 hours then was concentrated in vacuo. The residue was dissolved in AcOEt and the organic phase was washed with a saturated NaHCO₃ aqueous solution. The aqueous phase was extracted with AcOEt and the combined organic phases were washed with saturated brine, dried over MgSO₄ and concentrated in vacuo. Purification of the residue by flash chromatography on silica gel (25 g, c-Hexane/AcOEt: 50%) gave 1,1-dimethylethyl 3-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanoate (84 mg, 87%) as a pale yellow oil which solidified on standing.

LCMS: Retention time 1.12 min; [M+H]⁺=518, 520

### PREPARATION 6 ALTERNATIVE PROCEDURE

### 1,1-Dimethylethyl 3-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanoate

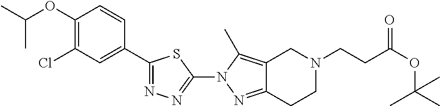

2-(5-{3-chloro-4-[(1-methylethyl)oxy]phenyl}-1,3,4-thiadiazol-2-yl)-3-methyl-4,5,6,7-tetrahydro-2H-pyrazolo[4,3-c]pyridine (Example 1) (31.4 g, 72.5 mmol) and 1,1-dimethylethyl 2-propenoate (13.93 g, 109 mmol) and 2,3,4,6,7,8,9,10-octahydropyrimido[1,2-a]azepine (33.1 g, 217 mmol) were combined in DMF and stirred overnight at room temperature. The mixture was diluted with water (700 ml), giving a dense white solid which was collected by filtration and washed with water (100 ml). Product was dissolved in hot EtOAc (1 L) with some difficulty, then washed with water (2×300 ml), dried and evaporated to give 1,1-dimethylethyl 3-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanoate (36.8 g, 98%)

LCMS: Retention time 1.13 min; [M+H]⁺=518, 520

1H NMR(CHLOROFORM-d, 400 MHz): δ (ppm) 7.98 (d, J=2.3 Hz, 1H), 7.76 (dd, J=8.6, 2.3 Hz, 1H), 7.03 (d, J=8.8 Hz, 1H), 4.68 (spt, J=6.1 Hz, 1H), 3.51 (s, 2H), 2.92 (t, J=7.2 Hz, 2H), 2.79-2.87 (m, 4H), 2.66 (s, 3H), 2.53 (t, J=7.3 Hz, 2H), 1.47 (s, 9H), 1.44 (d, J=6.1 Hz, 6H)

### PREPARATION 7

### 5-(5-Amino-1,3,4-thiadiazol-2-yl)-2-[(1-methylethyl)oxy]benzonitrile

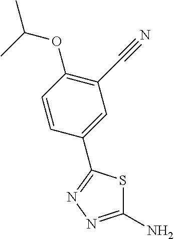

A flask was charged with 3-cyano-4-[(1-methylethyl)oxy]benzoic acid (CAS#: 258273-31-3, commercially available from Boaopharma, 20.9 g, 102 mmol) and hydrazinecarbothioamide (CAS#: 79-19-6, commercially available from Aldrich, 13.9 g, 153 mmol) then phosphorus oxychloride (CAS#: 10025-87-3, commercially available from Aldrich, 90 g, 587 mmol) was added. The resulting mixture was stirred at 90° C. for 3 hours then cooled to room temperature and added very carefully in small portions to a 5M NaOH aqueous solution cooled with an ice bath such that the temperature never rose above 35° C. The resulting mixture was basified to pH 10 (using a 5M NaOH aqueous solution) then stirred 30 minutes. The precipitate formed was collected by filtration and dissolved in DCM (1 l) and MeOH (50 ml). The organic phase was washed with water (500 ml), dried and concentrated in vacuo to give 5-(5-amino-1,3,4-thiadiazol-2-yl)-2-[(1-methylethyl)oxy]benzonitrile (26.3 g, 99% yield) as pale yellow solid which was used in the next step without further purification.

LCMS: Retention time 0.84 min; [M+H]⁺=261.13

### PREPARATION 8

### 5-(5-Bromo-1,3,4-thiadiazol-2-yl)-2-[(1-methylethyl)oxy]benzonitrile

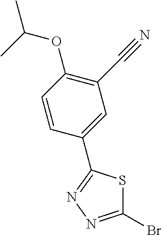

Cupric bromide (19.63 g, 88 mmol) and t-butyl nitrite (10.44 ml, 88 mmol) were dissolved in CH₃CN (400 ml) and the resulting mixture was stirred for 10 minutes at room temperature. 5-(5-Amino-1,3,4-thiadiazol-2-yl)-2-[(1-methylethyl)oxy]benzo nitrile (Preparation 7) (13 g, 40.0 mmol) was then added in small portions over 30 minutes. The resulting mixture was stirred for 1 hour at room temperature, at 70° C. for 2 hours then cooled and concentrated in vacuo. The residue was dissolved in AcOEt (600 ml) and MeOH (50 ml) and stirred at reflux for 1 hour. The insoluble material was filtered through a silica pad and washed with AcOEt (2×200 ml). The combined organic phases were washed with a 1M HCl aqueous solution (300 ml), dried and concentrated in vacuo. Material loaded onto silica cartridge (330 g) in DCM (100 ml) and purified by flash chromatography (c-Hexane/AcOEt: 0 to 100% gradient) to give 5-(5-bromo-1,3,4-thiadiazol-2-yl)-2-[(1-methylethyl)oxy]benzonitrile as a pale yellow solid (8.8 g, 67.9%)

LCMS: Retention time 1.14 min; [M+H]⁺=324, 326

### PREPARATION 9

### 5-(5-Hydrazino-1,3,4-thiadiazol-2-yl)-2-[(1-methylethyl)oxy]benzonitrile

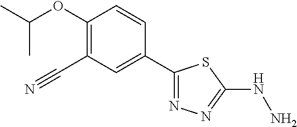

A solution of 5-(5-bromo-1,3,4-thiadiazol-2-yl)-2-[(1-methylethyl)oxy]benzonitrile (Preparation 8) (0.5 g, 1.542 mmol) and hydrazine hydrate (0.240 ml, 7.71 mmol) in i-PrOH (10 ml) was stirred at 100° C. under nitrogen. After 2 hours, the reaction mixture had solidified and was cooled to room temperature. DCM (10 ml) was added and the resulting solution was washed with a saturated NaHCO₃ aqueous solution. The two layers were separated using a phase separator cartridge and the organic phase was concentrated in vacuo. Purification of the residue by flash chromatography on silica gel (DCM/MeOH: 2.5 to 10% gradient) gave 5-(5-hydrazino-1,3,4-thiadiazol-2-yl)-2-[(1-methylethyl)oxy]benzonitrile (272 mg, 64%) as a white solid.

LCMS: Retention time 0.84 min; [M+H]⁺=276

### PREPARATION 9 ALTERNATIVE PROCEDURE

### 5-(5-Hydrazino-1,3,4-thiadiazol-2-yl)-2-[(1-methylethyl)oxy]benzonitrile

N12189-69-1

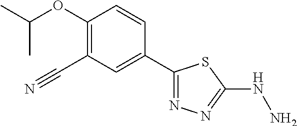

5-(5-bromo-1,3,4-thiadiazol-2-yl)-2-[(1-methylethyl)oxy]benzonitrile (Preparation 8) (7.8 g, 24.06 mmol) was suspended in isopropanol (60 mL) under nitrogen. Hydrazine hydrate (7.55 mL, 241 mmol) was added and the mixture heated to 105° C. overnight. The solvent was evaporated in vacuo, water added and the solid product collected by filtration to give 5-(5-hydrazino-1,3,4-thiadiazol-2-yl)-2-[(1-methylethyl)oxy]benzonitrile as pale green solid (6.2 g, 94%)

LCMS: Retention time 0.85 min; [M+H]⁺=276

### PREPARATION 10

### 1,1-Dimethylethyl 2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridine-5-carboxylate

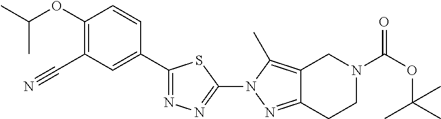

A mixture of 5-(5-hydrazino-1,3,4-thiadiazol-2-yl)-2-[(1-methylethyl)oxy]benzonitrile (Preparation 9) (274 mg, 0.995 mmol) and 1,1-dimethylethyl 3-acetyl-4-oxo-1-piperidinecarboxylate (Preparation 4) (240 mg, 0.995 mmol) in N,N-dimethylacetamide (5 ml) was stirred at 150° C. for 1 hour under microwave irradiation then cooled to room temperature and concentrated in vacuo. Purification of the residue by flash chromatography on silica gel (25 g, c-Hexane/AcOEt: 10 to 50% gradient) gave 1,1-dimethylethyl 2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridine-5-carboxylate (165 mg, 34%) as a white solid

LCMS: Retention time 1.42 min; [M+H]⁺=480.9

### PREPARATION 11

### 1,1-Dimethylethyl 3-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanoate

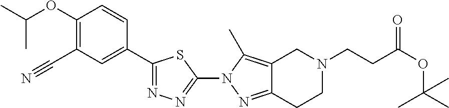

DBU (0.053 ml, 0.355 mmol) was added to a solution of 2-[(1-methylethyl)oxy]-5-[5-(3-methyl-4,5,6,7-tetrahydro-2H-pyrazolo[4,3-c]pyridin-2-yl)-1,3,4-thiadiazol-2-yl]benzonitrile (Example 3) (45 mg, 0.118 mmol) and 1,1-dimethylethyl 2-propenoate (0.086 ml, 0.591 mmol) in DMF (5 ml) at room temperature under nitrogen and the resulting mixture was stirred at this temperature overnight then concentrated in vacuo. The residue was partitioned between DCM and a saturated NaHCO₃ aqueous solution and the layers were separated. The organic phase was dried using a phase separator cartridge and concentrated in vacuo. Purification of the residue by flash chromatography on silica gel (12 g, c-Hexane/AcOEt: 10 to 70% gradient) gave 1,1-dimethylethyl 3-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanoate (46 mg, 76%) as a yellow solid.

LCMS: Retention time 0.99 min; [M+H]⁺=509.0

### PREPARATION 12

1,1-Dimethylethyl 2-(5-{3-chloro-4-[(1-methylethyl)oxy]phenyl}-1,3,4-thiadiazol-2-yl)-2,4,6,7-tetrahydro-5H-pyrazolo[4,3-c]pyridine-5-carboxylate (Preparation 12a) and 1,1-dimethylethyl 1-(5-{3-chloro-4-[(1-methylethyl)oxy]phenyl}-1,3,4-thiadiazol-2-yl)-1,4,6,7-tetrahydro-5H-pyrazolo[4,3-c]pyridine-5-carboxylate (Preparation 12b)

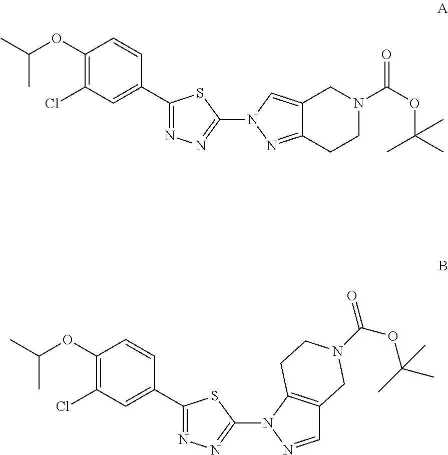

A mixture of 2-bromo-5-{3-chloro-4-[(1-methylethyl)oxy]phenyl}-1,3,4-thiadiazole (Preparation 2) (2 g, 5.99 mmol), 1,1-dimethylethyl 2,4,6,7-tetrahydro-5H-pyrazolo[4,3-c]pyridine-5-carboxylate (CAS#: 230301-11-8, commercially available from Bioblocks, 1.606 g, 7.19 mmol), copper(I) iodide (0.114 g, 0.599 mmol) and Cs₂CO₃ (3.91 g, 11.99 mmol) in DMF (20 ml) was stirred at 160° C. for 1 hour under microwave irradiation then partitioned between water (400 ml) and AcOEt (400 ml). The two layers were separated and the organic phase was washed with water (400 ml) then dried using a phase separation cartridge and concentrated in vacuo. Purification of the residue by flash chromatography on silica gel (65 g, c-Hexane/AcOEt: 7 to 20% gradient) over 20 column volumes of solvent. Appropriate fractions were combined and concentrated to give a mixture of isomers. Material further purified by flash chromatography on silica gel (40 g, c-Hexane/AcOEt: 10 to 20% gradient) over 18 column volumes of solvent. Appropriate fractions were combined and concentrated to give 1,1-dimethylethyl 1-(5-{3-chloro-4-[(1-methylethyl)oxy]phenyl}-1,3,4-thiadiazol-2-yl)-1,3a,4,6,7,7a-hexahydro-5H-pyrazolo[4,3-c]pyridine-5-carboxylate (Preparation 12b, 214 mg, 9%; LCMS: Retention time 1.58 min; [M+H]⁺=476, 478) as a cream solid and 1,1-dimethylethyl 2-(5-{3-chloro-4-[(1-methylethyl)oxy]phenyl}-1,3,4-thiadiazol-2-yl)-2,4,6,7-tetrahydro-5H-pyrazolo[4,3-c]pyridine-5-carboxylate (Preparation 12a, 247 mg, 8%; LCMS: Retention time 1.55 min; [M+H]⁺=476, 478) as a white solid.

### PREPARATION 13

Ethyl 4-[2-(5-{3-chloro-4-[(1-methylethyl)oxy]phenyl}-1,3,4-thiadiazol-2-yl)-2,4,6,7-tetrahydro-5H-pyrazolo[4,3-c]pyridin-5-yl]butanoate

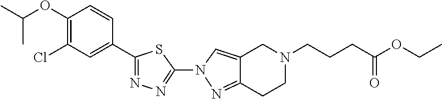

A mixture of 2-(5-{3-chloro-4-[(1-methylethyl)oxy]phenyl}-1,3,4-thiadiazol-2-yl)-4,5,6,7-tetrahydro-2H-pyrazolo[4,3-c]pyridine bis trifluoroacetic acid salt (Example 6) (100 mg, 0.166 mmol), ethyl 4-bromobutanoate (0.072 ml, 0.497 mmol) and K₂CO₃ (57.2 mg, 0.414 mmol) in DMF (3 ml) was stirred at 80° C. for 1 hour then more ethyl 4-bromobutanoate (0.024 ml, 0.166 mmol) was added. The resulting mixture was stirred at 80° C. for another 30 minutes then partitioned between AcOEt and water. The organic phase was washed with water and then passed through a phase separator cartridge and the organic phase was concentrated in vacuo. The residue was triturated with Et₂O and the solid formed was filtered off and dried under vacuum (ca 15 mbar) to give ethyl 4-[2-(5-{3-chloro-4-[(1-methylethyl)oxy]phenyl}-1,3,4-thiadiazol-2-yl)-2,4,6,7-tetrahydro-5H-pyrazolo[4,3-c]pyridin-5-yl]butanoate (46 mg, 57%) as a cream solid.

LCMS: Retention time 1.04 min; [M+H]⁺=490, 492

### PREPARATION 14

### 1,1-Dimethylethyl 2-(5-{3-cyano-4-[(1-methylethyl)oxy]phenyl}-1,3,4-thiadiazol-2-yl)-2,4,6,7-tetrahydro-5H-pyrazolo[4,3-c]pyridine-5-carboxylate

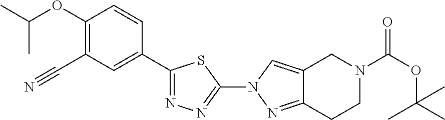

A mixture of 5-(5-bromo-1,3,4-thiadiazol-2-yl)-2-[(1-methylethyl)oxy]benzonitrile (Preparation 8) (700 mg, 2.159 mmol), 1,1-dimethylethyl 2,4,6,7-tetrahydro-5H-pyrazolo[4,3-c]pyridine-5-carboxylate (CAS#: 230301-11-8, commercially available from Bioblocks, 579 mg, 2.59 mmol), copper(I) iodide (41.1 mg, 0.216 mmol) and Cs₂CO₃ (1407 mg, 4.32 mmol) in DMF (14 ml) was stirred in at 160° C. for 60 minutes with microwave irradiation then a further 30 mins at 160° C. The mixture was partitioned between water (150 ml) and AcOEt (150 ml). The two layers were separated the aqueous phase was extracted with AcOEt (150 ml). The combined organic phases were dried using a phase separator cartridge and concentrated in vacuo. Residue purified by flash chromatography on silica gel (40 g, c-Hexane/AcOEt: 10 to 40% gradient) over 15 column volumes. Material purified by further flash chromatography on silica gel (25 g, c-Hexane/AcOEt: 10 to 35% gradient) over 15 column volumes, appropriate fractions combined to give 1,1-dimethylethyl 2-(5-{3-cyano-4-[(1-methylethyl)oxy]phenyl}-1,3,4-thiadiazol-2-yl)-2,4,6,7-tetrahydro-5H-pyrazolo[4,3-c]pyridine-5-carboxylate (315 mg, 31%) as a white solid.

LCMS: Retention time 1.39 min; [M+H]⁺=467.2

### PREPARATION 15

### Ethyl 3-[2-(5-{3-cyano-4-[(1-methylethyl)oxy]phenyl}-1,3,4-thiadiazol-2-yl)-2,4,6,7-tetrahydro-5H-pyrazolo[4,3-c]pyridin-5-yl]propanoate

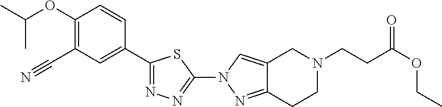

A mixture of 2-[(1-methylethyl)oxy]-5-[5-(4,5,6,7-tetrahydro-2H-pyrazolo[4,3-c]pyridin-2-yl)-1,3,4-thiadiazol-2-yl]benzonitrile trifluoroacetic acid salt (Example 11) (80 mg, 0.167 mmol), DBU (0.075 ml, 0.500 mmol) and ethyl 2-propenoate (0.090 ml, 0.833 mmol) was stirred in DMF (7 ml) at room temperature for overnight then was partitioned between AcOEt and water. The organic phase was washed with water then dried using a phase separator cartridge and concentrated in vacuo. The residue was triturated with Et₂O and the precipitate formed was filtered to give ethyl 3-[2-(5-{3-cyano-4-[(1-methylethyl)oxy]phenyl}-1,3,4-thiadiazol-2-yl)-2,4,6,7-tetrahydro-5H-pyrazolo[4,3-c]pyridin-5-yl]propanoate (19 mg, 24%) as a white solid.

LCMS: Retention time 0.96 min; [M+H]⁺=467.16

### PREPARATION 16

### Ethyl 4-[2-(5-{3-cyano-4-[(1-methylethyl)oxy]phenyl}-1,3,4-thiadiazol-2-yl)-2,4,6,7-tetrahydro-5H-pyrazolo[4,3-c]pyridin-5-yl]butanoate

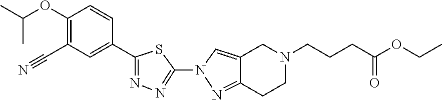

A mixture of 2-[(1-methylethyl)oxy]-5-[5-(4,5,6,7-tetrahydro-2H-pyrazolo[4,3-c]pyridin-2-yl)-1,3,4-thiadiazol-2-yl]benzonitrile trifluoroacetic acid salt (Example 11) (50 mg, 0.104 mmol), ethyl 4-bromobutanoate (0.045 ml, 0.312 mmol) and K₂O₃(36.0 mg, 0.260 mmol) in DMF (3 ml) was stirred at 80° C. for 1 hour and partitioned between AcOEt and water. The organic phase was washed with water then dried using a phase separator cartridge and concentrated in vacuo to give ethyl 4-[2-(5-{3-cyano-4-[(1-methylethyl)oxy]phenyl}-1,3,4-thiadiazol-2-yl)-2,4,6,7-tetrahydro-5H-pyrazolo[4,3-c]pyridin-5-yl]butanoate (164 mg, 107%) as a yellow oil which was used in the preparation of example 13 without further purification

LCMS: Retention time 0.93 min; [M+H]⁺=481.18

### PREPARATION 17

### 1,1-Dimethylethyl 4,5,7,8-tetrahydropyrazolo[3,4-c]azepine-6(2H)-carboxylate

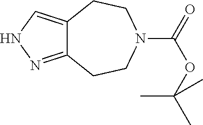

A solution of 2,4,5,6,7,8-hexahydropyrazolo[3,4-d]azepine hydrochloride (CAS#: 928774-98-5, commercially available from Allichem, 1 g, 5.76 mmol) in DMF (6 ml) and water (2 ml) at 0° C. was treated with di-tert-butyl dicarbonate (1.337 ml, 5.76 mmol) and a 2N NaOH aqueous solution (2.88 ml, 5.76 mmol) The resulting mixture was vigorously stirred for 2 hours and allowed to warm to room temperature then concentrated in vacuo. The residue was partitioned between DCM and a saturated NaHCO₃ aqueous solution and the layers were separated. The organic phase was dried using a phase separator cartridge and concentrated in vacuo to give 1,1-dimethylethyl 4,5,7,8-tetrahydropyrazolo[3,4-d]azepine-6(2H)-carboxylate (1.25 g, 88%) as a yellow oil which was used in the next step without further purification.

### PREPARATION 18

1,1-Dimethylethyl 2-(5-{3-chloro-4-[(1-methylethyl)oxy]phenyl}-1,3,4-thiadiazol-2-yl)-4,5,7,8-tetrahydropyrazolo[3,4-c]azepine-6(2H)-carboxylate (Preparation 18a) and 1,1-dimethylethyl 1-(5-{3-chloro-4-[(1-methylethyl)oxy]phenyl}-1,3,4-thiadiazol-2-yl)-4,5,7,8-tetrahydropyrazolo[3,4-c]azepine-6(1H)-carboxylate (Preparation 18b)

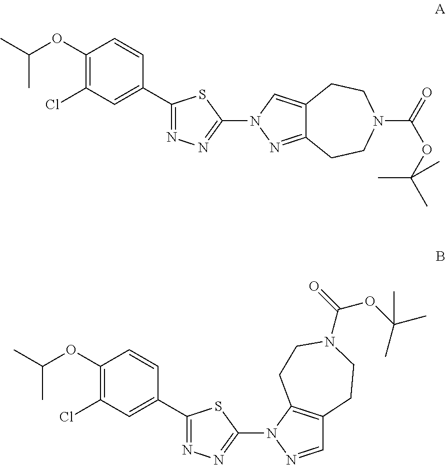

A mixture of 2-bromo-5-{3-chloro-4-[(1-methylethyl)oxy]phenyl}-1,3,4-thiadiazole (Preparation 2) (236 mg, 0.708 mmol), Cs₂CO₃ (308 mg, 0.944 mmol), copper(I) iodide (27.0 mg, 0.142 mmol) and 1,1-dimethylethyl 4,5,7,8-tetrahydropyrazolo[3,4-d]azepine-6(2H)-carboxylate (Preparation 17) (112 mg, 0.472 mmol) in DMF (2 ml) was stirred at 150° C. for 1 hour under microwave irradiation then cooled to room temperature and concentrated in vacuo. The residue was partitioned between AcOEt and a saturated NaHCO₃ aqueous solution and the two layers were separated. The aqueous phase was extracted with AcOEt and the combined organic phases were washed with brine, dried over Na₂SO₄ and concentrated in vacuo. Purification of the residue by flash chromatography on silica gel (25 g, c-Hexane/AcOEt: 3 to 50% gradient) gave a mixture (2:1 ratio by 1H NMR) of 1,1-dimethylethyl 2-(5-{3-chloro-4-[(1-methylethyl)oxy]phenyl}-1,3,4-thiadiazol-2-yl)-4,5,7,8-tetrahydropyrazolo[3,4-d]azepine-6(2H)-carboxylate (Preparation 18a) and 1,1-dimethylethyl 1-(5-{3-chloro-4-[(1-methylethyl)oxy]phenyl}-1,3,4-thiadiazol-2-yl)-4,5,7,8-tetrahydropyrazolo[3,4-d]azepine-6(1H)-carboxylate (Preparation 18b) (93 mg, 40%) as an off white solid which was used in the next step without further purification.

LCMS: Retention time 1.58 min; [M+H]⁺=490, 492

### PREPARATION 19

2-(5-{3-Chloro-4-[(1-methylethyl)oxy]phenyl}-1,3,4-thiadiazol-2-yl)-2,4,5,6,7,8-hexahydropyrazolo[3,4-d]azepine (Preparation 19a) and 1-(5-{3-chloro-4-[(1-methylethyl)oxy]phenyl}-1,3,4-thiadiazol-2-yl)-1,4,5,6,7,8-hexahydropyrazolo[3,4-d]azepine (Preparation 19b)

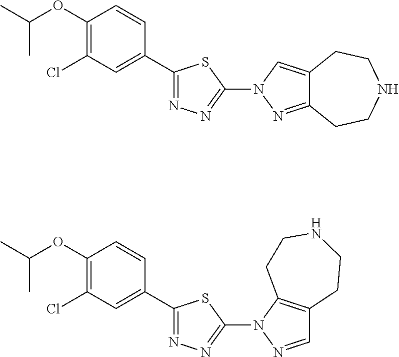

A suspension of 1,1-dimethylethyl 2-(5-{3-chloro-4-[(1-methylethyl)oxy]phenyl}-1,3,4-thiadiazol-2-yl)-4,5,7,8-tetrahydropyrazolo[3,4-d]azepine-6(2H)-carboxylate (Preparation 18a) and 1,1-dimethylethyl 1-(5-{3-chloro-4-[(1-methylethyl)oxy]phenyl}-1,3,4-thiadiazol-2-yl)-4,5,7,8-tetrahydropyrazolo[3,4-c]azepine-6(1H)-carboxylate (Preparation 18b) (2:1 ratio) (93 mg, 0.190 mmol) in 1,4-dioxane (2 ml) at room temperature was treated with a 4N HCl solution in 1,4-dioxane (1 ml, 4.00 mmol) and the resulting mixture was stirred at this temperature for 2 hours. More of a 4N HCl solution in 1,4-dioxane (2 ml, 8 mmol) was added and the resulting mixture was stirred for another 3.5 hours. More of a 4N HCl solution in 1,4-dioxane (1 ml, 4 mmol) was added again and the mixture was stirred for 48 hours at room temperature then diluted with MeOH (10 ml), loaded onto a SCX cartridge (20 g), then eluted sequentially with MeOH (75 ml) and a 2N NH₃ solution in MeOH (75 ml). The ammonia fractions were concentrated in vacuo to give a mixture (4:3 ratio by 1H NMR) of 2-(5-{3-Chloro-4-[(1-methylethyl)oxy]phenyl}-1,3,4-thiadiazol-2-yl)-2,4,5,6,7,8-hexahydropyrazolo[3,4-d]azepine (Preparation 19a) and 1-(5-{3-chloro-4-[(1-methylethyl)oxy]phenyl}-1,3,4-thiadiazol-2-yl)-1,4,5,6,7,8-hexahydropyrazolo[3,4-c]azepine (Preparation 19b) (80 mg, 108%) as a colourless foam which was used in the next step without further purification.

### PREPARATION 20 methyl 4-[2-(5-{3-chloro-4-[(1-methylethyl)oxy]phenyl}-1,3,4-thiadiazol-2-yl)-4,5,7,8-tetrahydropyrazolo[3,4-d]azepin-6(2H)-yl]butanoate

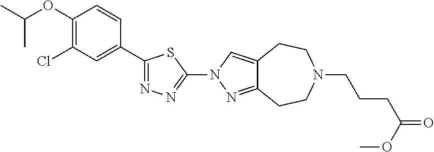

A mixture of 2-(5-{3-chloro-4-[(1-methylethyl)oxy]phenyl}-1,3,4-thiadiazol-2-yl)-2,4,5,6,7,8-hexahydropyrazolo[3,4-d]azepine (Preparation 19a) and 1-(5-{3-chloro-4-[(1-methylethyl)oxy]phenyl}-1,3,4-thiadiazol-2-yl)-1,4,5,6,7,8-hexahydropyrazolo[3,4-d]azepine (Preparation 19b) (4:3 ratio) (80 mg, 0.205 mmol), ethyl 4-bromobutanoate (0.035 ml, 0.246 mmol) and K₂CO₃ (56.7 mg, 0.410 mmol) in DMF (3.5 ml) was stirred at 100° C. under nitrogen for 2.5 hours then cooled to room temperature and concentrated in vacuo. The residue was dissolved in DCM (20 ml) and the organic phase was washed with water (20 ml). The layers were separated and the organic phase was dried using a phase separator cartridge, and concentrated in vacuo. Purification of the residue by Reversed Phase Mass Directed Preparative HPLC using a Zorbax SB Phenyl Column (7.0 um, 150×21.2 mmID) over 18 injections (100 uL, DMSO/MeOH 1:99, 4.7 mg loading) using a gradient 30-60B % over 24 mins at a flow rate of 20 mL/min at ambient temperature (Mobile Phase ‘A’: 0.1% v/v TFA in Water; Mobile Phase ‘B’: MeCN+0.1% v/v of TFA) Concentration of the appropriate fractions gave methyl 4-[2-(5-{3-chloro-4-[(1-methylethyl)oxy]phenyl}-1,3,4-thiadiazol-2-yl)-4,5,7,8-tetrahydropyrazolo[3,4-d]azepin-6(2H)-yl]butanoate (23 mg, 23%) as a colourless foam.

LCMS: Retention time 1.11 min; [M+H]⁺=490.2

### PREPARATION 21

### 5-{5-[5-(2,2-dimethyl-1,3-dioxan-5-yl)-3-methyl-4,5,6,7-tetrahydro-2H-pyrazolo[4,3-c]pyridin-2-yl]-1,3,4-thiadiazol-2-yl}-2-[(1-methylethyl)oxy]benzonitrile

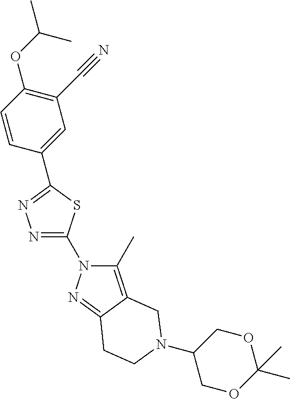

A mixture of 2-[(1-Methylethyl)oxy]-5-[5-(3-methyl-4,5,6,7-tetrahydro-2H-pyrazolo[4,3-c]pyridin-2-yl)-1,3,4-thiadiazol-2-yl]benzonitrile (Example 3a) (245 mg, 0.5 mmol), 2,2-dimethyl-1,3-dioxan-5-one (322 mg, 2.5 mmol) and sodium triacetoxyborohydride (525 mg, 2.5 mmol) in dichloromethane (10 ml) was stirred at room temperature overnight. The reaction mixture was diluted with saturated NaHCO₃ (10 ml) and extracted with ethyl acetate (3×10 ml). The combined organics were dried and evaporated (245 mg, 100%). The residue was used without further purification in the preparation of Example 25a LCMS: Retention time 0.89 min; [M+H]⁺=495

### PREPARATION 22

### 1,1-dimethylethyl {2-[2-(5-{3-cyano-4-[(1-methylethyl)oxy]phenyl}-1,3,4-thiadiazol-2-yl)-3-methyl-2,4,6,7-tetrahydro-5H-pyrazol o[4,3-c]pyridin-5-yl]-2-oxoethyl}carbamate

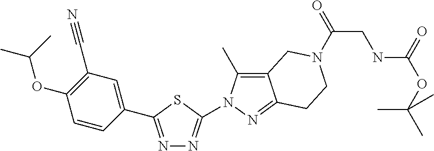

To N—BOC glycine (21.26 mg, 0.121 mmol) and HATU (57.7 mg, 0.152 mmol) in N,N-Dimethylformamide (DMF) (2 mL) was added DIPEA (0.053 mL, 0.303 mmol) and the reaction mixture stirred at room temperature for 10 mins. 2-[(1-methylethyl)oxy]-5-[5-(3-methyl-4,5,6,7-tetrahydro-2H-pyrazolo[4,3-c]pyridin-2-yl)-1,3,4-thiadiazol-2-yl]benzonitrile trifluoroacetate (Example 3 alternative preparation) (50 mg, 0.101 mmol) was added and the reaction mixture stirred at room temperature for a further 16 h. The residue was partitioned between dichloromethane (2×10 mL) and water (10 mL). The organics were combined and washed with water (5×10 mL), dried over a hydrophobic frit and evaporated to give the crude product 1,1-dimethylethyl {2-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-2-oxoethyl}carbamate (88 mg, 0.147 mmol) used without further purification in the preparation of Example 32.

LCMS: Retention time 1.23 min; [M+H]⁺=538

### PREPARATION 23

### 1,1-dimethylethyl [2-(5-{3-cyano-4-[(1-methylethyl)oxy]phenyl}-1,3,4-thiadiazol-2-yl)-3-methyl-2,4,6,7-tetrahydro-5H-pyrazol o[4,3-c]pyridin-5-yl]acetate

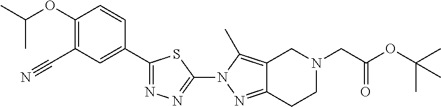

A mixture of 2-[(1-methylethyl)oxy]-5-[5-(3-methyl-4,5,6,7-tetrahydro-2H-pyrazolo[4,3-c]pyridin-2-yl)-1,3,4-thiadiazol-2-yl]benzonitrile (Example 3) (500 mg, 1.01 mmol), t-butyl bromoacetate (207 mg, 1.06 mmol) and potassium carbonate (419 mg, 3.03 mmol) in acetonitrile (15 mL) was stirred at 50° C. for 2 hours. LCMS showed complete reaction. The reaction mixture was cooled and diluted with water (15 mL). The mixture was extracted with ethyl acetate (2×20 mL). The combined extracts were dried and evaporated. The residue was chromatographed [0-3% methanol/dichloromethane] to give the product as a colourless foam (470 mg, 94%)

LCMS: Retention time 1.02 min; [M+H]⁺=495

### PREPARATION 24

### 2-(5-{3-chloro-4-[(1-methylethyl)oxy]phenyl}-1,3,4-thiadiazol-2-yl)-5-(2,2-dimethyl-1,3-dioxan-5-yl)-3-methyl-4,5,6,7-tetrahydro-2H-pyrazolo[4,3-c]pyridine

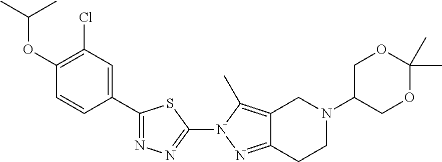

2,2-dimethyl-1,3-dioxan-5-one (0.660 g, 5.07 mmol) was added to 2-(5-{3-chloro-4-[(1-methylethyl)oxy]phenyl}-1,3,4-thiadiazol-2-yl)-3-methyl-4,5,6,7-tetrahydro-2H-pyrazolo[4,3-c]pyridine hydrochloride (Example 1b) (0.470 g, 1.102 mmol) in dry Dichloromethane (DCM) (5 mL) and the reaction was left to stir at room temperature for 30 min. Sodium triacetoxyborohydride (1.060 g, 5.00 mmol) was then added and the reaction was left to stir overnight. The reaction mixture was quenched with saturated solution of sodium bicarbonate (10 mL), extracted with DCM (3×10 mL) and then concentrated under reduced pressure to get a brownish yellow oil, on resting it became solid (829 mg). The solid was dissolved in a small amount of DCM, loaded onto a (100 g) silica cartridge and was eluted with 8 column volumes of ethyl acetate/cyclohexane gradient (30-80%) and 2 CV of 80% ethyl acetate/cyclohexane mixture and 5 CV of ethyl acetate/cyclohexane gradient (80-100%). The cartridge was again eluted with 6 CV of ethyl acetate/cyclohexane gradient (80-100%). The pure fractions collected were combined and concentrated under reduced pressure get a brownish yellow solid (374 mg, 57%).

LCMS: Retention time 1.02 min; [M+H]⁺=504, 506

### EXAMPLE 1a

### 2-(5-{3-Chloro-4-[(1-methylethyl)oxy]phenyl}-1,3,4-thiadiazol-2-yl)-3-methyl-4,5,6,7-tetrahydro-2H-pyrazolo[4,3-c]pyridine

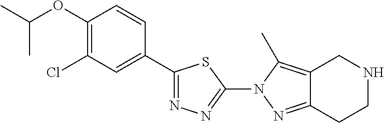

Trifluoroacetic acid (0.5 ml, 6.49 mmol) was added to a solution of 1,1-dimethylethyl 2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridine-5-carboxylate (Preparation 5) (245 mg, 0.500 mmol) in DCM (5 ml) at room temperature under nitrogen and the resulting mixture was stirred at this temperature for 1 hour. Further trifluoroacetic acid (1.0 ml, 12.98 mmol) was then added and the resulting mixture was stirred for a further 30 minutes then concentrated in vacuo. The residue was co-evaporated with DCM and dried under high vacuum overnight. The sample was loaded in DCM onto an SCX cartridge (50 g) then eluted with MeOH followed by a 2N NH₃ solution in MeOH. The appropriate fractions were combined and concentrated in vacuo to give 2-(5-{3-chloro-4-[(1-methylethyl)oxy]phenyl}-1,3,4-thiadiazol-2-yl)-3-methyl-4,5,6,7-tetrahydro-2H-pyrazolo[4,3-c]pyridine (180 mg, 92%) as a yellow oil.

LCMS: Retention time 0.93 min; [M+H]⁺=390.10

### EXAMPLE 1b

### 2-(5-{3-chloro-4-[(1-methylethyl)oxy]phenyl}-1,3,4-thiadiazol-2-yl)-3-methyl-4,5,6,7-tetrahydro-2H-pyrazolo[4,3-c]pyridine hydrochloride

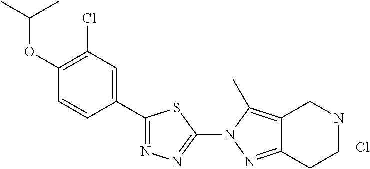

1,1-dimethylethyl 2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridine-5-carboxylate (35.5 g, 72.4 mmol) in DCM (5 mL) was added to a solution of HCl (181 mL, 724 mmol) in dioxan and the mixture was stirred for 30 min, then diluted with ether and the mixture stirred for 20 min. The suspension was filtered and the solid product collected by filtration to give 2-(5-{3-chloro-4-[(1-methylethyl)oxy]phenyl}-1,3,4-thiadiazol-2-yl)-3-methyl-4,5,6,7-tetrahydro-2H-pyrazolo[4,3-c]pyridine hydrochloride as a colourless solid (32.5 g, 95% yield)

LCMS: Method HpH Retention time 1.25 min; [M+H]⁺=390, 392

1H NMR (400 MHz, DMSO-d6) δ ppm 1.34 (d, J=6.1 Hz, 6H) 2.65 (s, 3H) 2.98 (t, J=6.1 Hz, 2H) 3.40-3.48 (m, 2H) 3.57 (s, 2H) 4.21 (s, 2H) 4.73-4.93 (m, 1H) 7.36 (d, J=8.8 Hz, 1H) 7.90 (dd, J=8.6, 2.3 Hz, 1H) 8.04 (d, J=2.3 Hz, 1H) 9.52 (br. s., 2H)

### EXAMPLE 2a

### 3-[2-(5-{3-Chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanoic acid trifluoroacetic acid salt

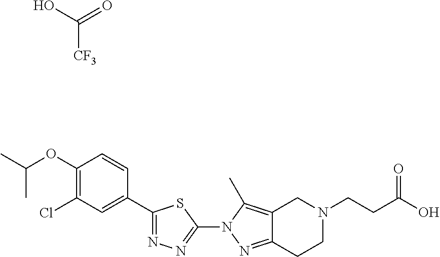

Trifluoroacetic acid (0.5 ml, 6.49 mmol) was added to a solution of 1,1-dimethylethyl 3-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanoate (Preparation 6) (84 mg, 0.162 mmol) in DCM (3 ml) at room temperature under nitrogen and the resulting mixture was stirred at this temperature for 5 hours, then diluted with DCM and concentrated in vacuo. The residue was triturated with a mixture of AcOEt and c-hexane and the solid formed was filtered off and dried under vacuum to give 3-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanoic acid trifluoroacetic acid salt (63 mg, 68%) as a pale yellow solid.

LCMS: Retention time 0.96 min; [M+H]⁺=462.14

### EXAMPLE 2b

### 3-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanoic acid hydrochloride

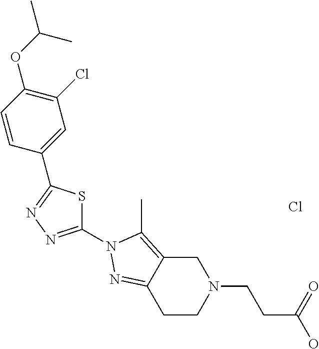

1,1-dimethylethyl 3-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanoate (Preparation 6) (36 g, 69.5 mmol) was suspended in a mixture of THF (500 mL) and 2M HCl (600 mL) and the thick suspension was heated at 50° C. for 18 h, giving a white suspension. The mixture was cooled to room temperature then filtered and the solid washed with water (2×50 mL) to give 3-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanoic acid hydrochloride as a colourless solid (30.35 g, 88%)

LCMS: Retention time 0.90 min; [M+H]⁺=462, 464

1H NMR (400 MHz, DMSO-d₆) δ ppm 8.04 (1H, d, J=2.5 Hz) 7.90 (1H, dd, J=9.0, 2.5 Hz) 7.36 (1H, d, J=9.0 Hz) 4.83 (1H, spt, J=6.0 Hz) 3.57-4.70 (4H, m) 3.40-3.52 (2H, m) 3.08 (2H, t, J=6.0 Hz) 2.91 (2H, t, J=7.5 Hz) 2.65 (3H, s) 1.34 (6H, d, J=6.0 Hz)

### EXAMPLE 3a

### 2-[(1-Methylethyl)oxy]-5-[5-(3-methyl-4,5,6,7-tetrahydro-2H-pyrazolo[4,3-c]pyridin-2-yl)-1,3,4-thiadiazol-2-yl]benzonitrile

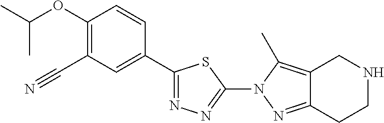

Trifluoroacetic acid (0.503 ml, 6.52 mmol) was added to a solution of 1,1-dimethylethyl 2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridine-5-carboxylate (Preparation 10) (165 mg, 0.343 mmol) in DCM (2 ml) at room temperature under nitrogen and the resulting mixture was stirred at this temperature for 2 hours then diluted with DCM (10 ml) and concentrated in vacuo. The residue was loaded on a SCX cartridge (10 g) then eluted with MeOH followed by a 2N NH₃ solution in MeOH. The combined ammonia fractions were concentrated in vacuo to give 2-[(1-methylethyl)oxy]-5-[5-(3-methyl-4,5,6,7-tetrahydro-2H-pyrazolo[4,3-c]pyridin-2-yl)-1,3,4-thiadiazol-2-yl]benzonitrile (119 mg, 91%) as a yellow solid.

LCMS: Retention time 0.84 min; [M+H]⁺=381.18

### EXAMPLE 3b

### 2-[(1-methylethyl)oxy]-5-[5-(3-methyl-4,5,6,7-tetrahydro-2H-pyrazolo[4,3-c]pyridin-2-yl)-1,3,4-thiadiazol-2-yl]benzonitrile trifluoroacetate

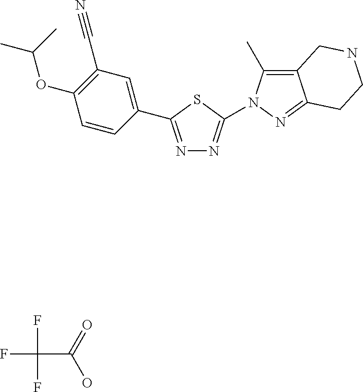

TFA (20 mL, 260 mmol) was added to a solution of 1,1-dimethylethyl 2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridine-5-carboxylate (Preparation 10) (5.3 g, 11.03 mmol) in DCM (200 mL) and the solution was stirred for 3 h, then evaporated in vacuo and the residue dissolved in methanol (25 mL) and diluted with ether (75 mL). The resulting suspension was stirred for 20 min, then filtered to give 2-[(1-methylethyl)oxy]-5-[5-(3-methyl-4,5,6,7-tetrahydro-2H-pyrazolo[4,3-c]pyridin-2-yl)-1,3,4-thiadiazol-2-yl]benzonitrile trifluoroacetate as a cream coloured solid (4.30 g, 79%)

LCMS: Retention time 1.08 min; [M+H]⁺=381

1H NMR (DMSO-d6, 400 MHz): δ (ppm) 9.10-9.27 (m, 2H), 8.34 (d, J=2.3 Hz, 1H), 8.23-8.27 (m, 1H), 7.48 (d, J=9.3 Hz, 1H), 4.87-5.01 (m, 1H), 4.24 (s, 2H), 3.44-3.51 (m, 2H), 2.94-3.02 (m, 2H), 2.66 (s, 3H), 1.37 (d, J=6.1 Hz, 6H)

### EXAMPLE 4

### 3-[2-(5-{3-Cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanoic acid trifluoroacetic acid salt

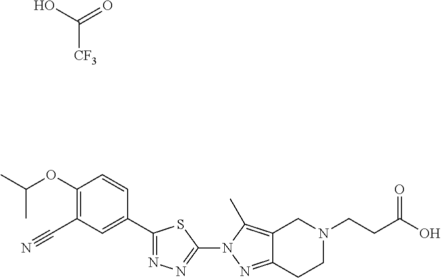

Trifluoroacetic acid (0.348 ml, 4.52 mmol) was added to a solution of 1,1-dimethylethyl 3-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanoate (Preparation 11) (46 mg, 0.090 mmol) in DCM (2 ml) at room temperature under nitrogen and the resulting mixture was stirred at this temperature for 7 hours, then diluted with DCM and concentrated in vacuo. Trituration of the residue with c-Hexane/AcOEt 1:1 gave 3-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanoic acid trifluoroacetic acid salt (25 mg, 48%) as an off white solid.

LCMS: Retention time 0.86 min; [M+H]⁺=453.0

### EXAMPLE 5

### 3-[2-(5-{3-Cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanamide

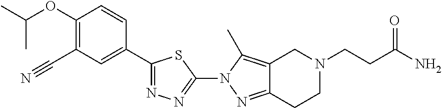

2-[(1-Methylethyl)oxy]-5-[5-(3-methyl-4,5,6,7-tetrahydro-2H-pyrazolo[4,3-c]pyridin-2-yl)-1,3,4-thiadiazol-2-yl]benzonitrile (Example 3) (25 mg, 0.066 mmol) and 2-propenamide (4.67 mg, 0.066 mmol) were dissolved in acetonitrile (1.5 ml). Silica gel (500 mg, 8.32 mmol) was added and the resulting mixture was stirred at 60° C. under nitrogen for 5 hours, then at room temperature for 16 hours. More 2-propenamide (1 mg, 0.014 mmol) was added and the resulting mixture was stirred at 60° C. under nitrogen for 4 hours, then filtered through a celite column. The insoluble material was washed with DCM, MeOH and AcOEt and the combined organic phases were concentrated in vacuo. Purification of the residue by MDAP (Method formate) gave 3-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanamide (25 mg, 84%) as a pale yellow solid.

LCMS: Retention time 0.79 min; [M+H]⁺=452.0

### EXAMPLE 6a

### 2-(5-{3-Chloro-4-[(1-methylethyl)oxy]phenyl}-1,3,4-thiadiazol-2-yl)-4,5,6,7-tetrahydro-2H-pyrazolo[4,3-c]pyridine bis trifluoroacetic acid salt

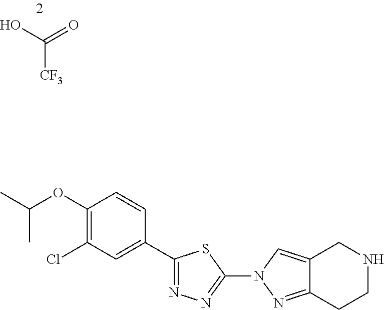

Trifluoroacetic acid (2.0 ml) was added to a solution of 1,1-dimethylethyl 2-(5-{3-chloro-4-[(1-methylethyl)oxy]phenyl}-1,3,4-thiadiazol-2-yl)-2,4,6,7-tetrahydro-5H-pyrazolo[4,3-c]pyridine-5-carboxylate (Preparation 12a) (970 mg, 2.038 mmol) in DCM (20 ml) and the resulting mixture was stirred at room temperature for 3 hours then concentrated in vacuo. The residue was triturated with Et₂O and the precipitate formed was filtered off and dried under vacuum (ca 15 mbar) to give 2-(5-{3-chloro-4-[(1-methylethyl)oxy]phenyl}-1,3,4-thiadiazol-2-yl)-4,5,6,7-tetrahydro-2H-pyrazolo[4,3-c]pyridine bis trifluoroacetic acid salt (953 mg, 77%) as a white solid.

LCMS: Retention time 0.94 min; [M+H]⁺=376.16

### EXAMPLE 6b

### 2-(5-{3-Chloro-4-[(1-methylethyl)oxy]phenyl}-1,3,4-thiadiazol-2-yl)-4,5,6,7-tetrahydro-2H-pyrazolo[4,3-c]pyridine trifluoroacetic acid salt

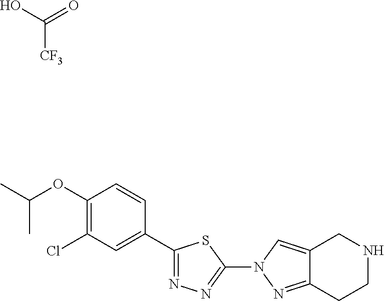

Trifluoroacetic acid (0.142 ml, 1.838 mmol) was added to a solution of 1,1-dimethylethyl 2-(5-{3-chloro-4-[(1-methylethyl)oxy]phenyl}-1,3,4-thiadiazol-2-yl)-2,4,6,7-tetrahydro-5H-pyrazolo[4,3-c]pyridine-5-carboxylate (Preparation 12a) (35 mg, 0.074 mmol) in DCM (2 ml) and the resulting mixture was stirred at room temperature for 16 hours then concentrated in vacuo to give 2-(5-{3-chloro-4-[(1-methylethyl)oxy]phenyl}-1,3,4-thiadiazol-2-yl)-4,5,6,7-tetrahydro-2H-pyrazolo[4,3-c]pyridine trifluoroacetic acid salt (35 mg, 97%) as a pale yellow solid.

LCMS: Retention time 1.04 min; [M+H]⁺=376.20

### EXAMPLE 7

### 4-[2-(5-{3-Chloro-4-[(1-methylethyl)oxy]phenyl}-1,3,4-thiadiazol-2-yl)-2,4,6,7-tetrahydro-5H-pyrazolo[4,3-c]pyridin-5-yl]butanoic acid

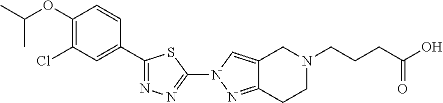

A solution of ethyl 4-[2-(5-{3-chloro-4-[(1-methylethyl)oxy]phenyl}-1,3,4-thiadiazol-2-yl)-2,4,6,7-tetrahydro-5H-pyrazolo[4,3-c]pyridin-5-yl]butanoate (Preparation 13) (45 mg, 0.092 mmol) in THF) (2 ml) and water (1.0 ml) was treated with LiOH (4.4 mg, 0.184 mmol) and the resulting mixture was stirred at room temperature for 2 hours, then dissolved with AcOEt. The organic phase was washed sequentially with a saturated NH₄Cl aqueous solution then water. The organic phase and water were separated using a phase separator cartridge and the organic phase was concentrated in vacuo to give 4-[2-(5-{3-chloro-4-[(1-methylethyl)oxy]phenyl}-1,3,4-thiadiazol-2-yl)-2,4,6,7-tetrahydro-5H-pyrazolo[4,3-c]pyridin-5-yl]butanoic acid (26 mg, 61%) as a white solid.

LCMS: Retention time 0.97 min; [M+H]⁺=462.21

### EXAMPLE 8

### 3-[2-(5-{3-Chloro-4-[(1-methylethyl)oxy]phenyl}-1,3,4-thiadiazol-2-yl)-2,4,6,7-tetrahydro-5H-pyrazolo[4,3-c]pyridin-5-yl]propanoic acid

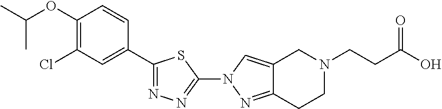

A mixture of 2-(5-{3-chloro-4-[(1-methylethyl)oxy]phenyl}-1,3,4-thiadiazol-2-yl)-4,5,6,7-tetrahydro-2H-pyrazolo[4,3-c]pyridine trifluoroacetic acid salt (Example 6) (32 mg, 0.085 mmol), 1,1-dimethylethyl 2-propenoate (0.025 ml, 0.170 mmol) and triethylamine (0.036 ml, 0.255 mmol) in n-BuOH (1.5 ml) and THF (0.5 ml) was stirred at 100° C. for 2 hours under microwave irradiation then cooled to room temperature and loaded onto a SCX column (5 g), then eluted sequentially with MeOH (100 ml) and a 1M NH₃ solution in MeOH (100 ml). The ammonia fractions were concentrated in vacuo and the residue dissolved in DCM (1 ml) and treated with trifluoroacetic acid (9.71 mg, 0.085 mmol). The resulting mixture was stirred at room temperature for 1 hour then concentrated in vacuo. Purification of the residue by MDAP (Method Formate) gave 3-[2-(5-{3-chloro-4-[(1-methylethyl)oxy]phenyl}-1,3,4-thiadiazol-2-yl)-2,4,6,7-tetrahydro-5H-pyrazolo[4,3-c]pyridin-5-yl]propanoic acid (8 mg, 21%) as a white oily solid.

LCMS: Retention time 0.96 min; [M+H]⁺=448.2

### EXAMPLE 9

### 3-[2-(5-{3-Chloro-4-[(1-methylethyl)oxy]phenyl}-1,3,4-thiadiazol-2-yl)-2,4,6,7-tetrahydro-5H-pyrazolo[4,3-c]pyridin-5-yl]-1-propanol

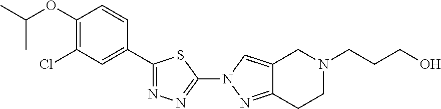

A solution of 2-(5-{3-chloro-4-[(1-methylethyl)oxy]phenyl}-1,3,4-thiadiazol-2-yl)-4,5,6,7-tetrahydro-2H-pyrazolo[4,3-c]pyridine bis trifluoroacetic acid salt (Example 6) (30 mg, 0.050 mmol) in DMF (1.5 ml) at room temperature under nitrogen was treated with sodium hydride (60% w/w in mineral oil, 6.95 mg, 0.174 mmol) and the resulting mixture was stirred at this temperature for 30 minutes. 3-Bromo-1-propanol (4.78 μL, 0.055 mmol) was then added and the resulting mixture stirred at this temperature for 40 hours. More 3-bromo-1-propanol (4.34 μL, 0.050 mmol) was added and the resulting mixture was stirred at room temperature for another 16 hours then diluted with AcOEt. The organic phase was washed sequentially with a saturated NaHCO₃ aqueous solution and brine, dried using a phase separator cartridge and concentrated in vacuo. The residue was triturated with Et₂O and the solid formed was filtered off and dried under vacuum (ca 15 mbar) to give 3-[2-(5-{3-chloro-4-[(1-methylethyl)oxy]phenyl}-1,3,4-thiadiazol-2-yl)-2,4,6,7-tetrahydro-5H-pyrazolo[4,3-c]pyridin-5-yl]-1-propanol (1.1 mg, 5%) as a yellow solid.

LCMS: Retention time 0.88 min; [M+H]⁺=434.10

### EXAMPLE 10

### 2-(5-{3-Chloro-4-[(1-methylethyl)oxy]phenyl}-1,3,4-thiadiazol-2-yl)-5-[2-(methylsulfonyl)ethyl]-4,5,6,7-tetrahydro-2H-pyrazolo[4,3-c]pyridine

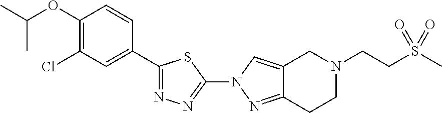

A mixture of 2-(5-{3-chloro-4-[(1-methylethyl)oxy]phenyl}-1,3,4-thiadiazol-2-yl)-4,5,6,7-tetrahydro-2H-pyrazolo[4,3-c]pyridine bis trifluoroacetic acid salt (Example 6) (30 mg, 0.050 mmol), K₂CO₃ (14.42 mg, 0.104 mmol) and (methylsulfonyl)ethene (10.88 μL, 0.124 mmol) in MeOH (2 ml) was stirred at room temperature for 48 hours. More (methylsulfonyl)ethene (8.7 μL, 0.10 mmol) was added and the resulting mixture was stirred at room temperature for 4 hours then concentrated in vacuo. The residue was triturated with MeOH and the precipitate formed was filtered off and dried under vacuum (ca 15 mbar) to give 2-(5-{3-chloro-4-[(1-methylethyl)oxy]phenyl}-1,3,4-thiadiazol-2-yl)-5-[2-(methylsulfonyl)ethyl]-4,5,6,7-tetrahydro-2H-pyrazolo[4,3-c]pyridine (12.7 mg, 53%) as a pale yellow solid.

LCMS: retention time 0.99 min; [M+H]⁺=482, 484

### EXAMPLE 11

### 2-[(1-Methylethyl)oxy]-5-[5-(4,5,6,7-tetrahydro-2H-pyrazolo[4,3-c]pyridin-2-yl)-1,3,4-thiadiazol-2-yl]benzonitrile trifluoroacetic acid salt

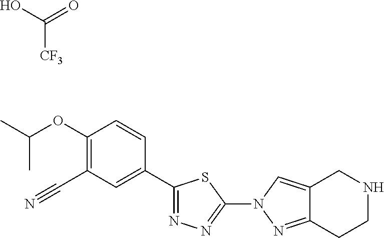

A solution of 1,1-dimethylethyl 2-(5-{3-cyano-4-[(1-methylethyl)oxy]phenyl}-1,3,4-thiadiazol-2-yl)-2,4,6,7-tetrahydro-5H-pyrazolo[4,3-c]pyridine-5-carboxylate (Preparation 14) (315 mg, 0.675 mmol) in DCM (7 ml) at room temperature was treated with trifluoroacetic acid (1 ml) and the resulting mixture was stirred at this temperature for 1.5 hours then concentrated in vacuo. The residue was triturated with Et₂O and the precipitate formed was filtered off and dried under vacuum (ca 15 mbar) to give 2-[(1-methylethyl)oxy]-5-[5-(4,5,6,7-tetrahydro-2H-pyrazolo[4,3-c]pyridin-2-yl)-1,3,4-thiadiazol-2-yl]benzonitrile trifluoroacetic acid salt (291 mg, 90%) as a white solid.

LCMS: Retention time 0.85 min; [M+H]⁺=367

### EXAMPLE 12

### 3-[2-(5-{3-Cyano-4-[(1-methylethyl)oxy]phenyl}-1,3,4-thiadiazol-2-yl)-2,4,6,7-tetrahydro-5H-pyrazolo[4,3-c]pyridin-5-yl]propanoic acid

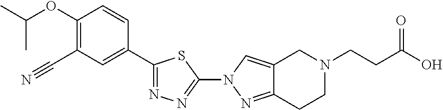

A solution of ethyl 3-[2-(5-{3-cyano-4-[(1-methylethyl)oxy]phenyl}-1,3,4-thiadiazol-2-yl)-2,4,6,7-tetrahydro-5H-pyrazolo[4,3-c]pyridin-5-yl]propanoate (Preparation 15) (18 mg, 0.039 mmol) in THF (1 ml) and water (0.5 ml) was treated at room temperature with LiOH (1.85 mg, 0.077 mmol) and the resulting mixture was stirred at this temperature for 2 hours. More LiOH (0.9 mg, 0.04 mmol) was added and the resulting mixture was stirred at room temperature for another 45 minutes. 1 extra eq of lithium hydroxide (0.924 mg, 0.039 mmol) was added and the mixture was left to stir at room temperature for 30 mins then was diluted with AcOEt. The organic phase was washed sequentially with a saturated NH₄Cl aqueous solution and water. The combined aqueous layers were extracted twice with DCM and the combined organic phases were dried using a phase separator cartridge and concentrated in vacuo to give 3-[2-(5-{3-cyano-4-[(1-methylethyl)oxy]phenyl}-1,3,4-thiadiazol-2-yl)-2,4,6,7-tetrahydro-5H-pyrazolo[4,3-c]pyridin-5-yl]propanoic acid (18 mg, 106%) as a white solid.

LCMS: Retention time 0.91 min; [M+H]⁺=439

### EXAMPLE 13

### 4-[2-(5-{3-Cyano-4-[(1-methylethyl)oxy]phenyl}-1,3,4-thiadiazol-2-yl)-2,4,6,7-tetrahydro-5H-pyrazolo[4,3-c]pyridin-5-yl]butanoic acid

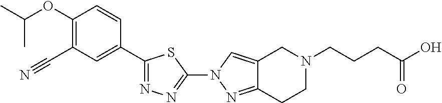

A solution of ethyl 4-[2-(5-{3-cyano-4-[(1-methylethyl)oxy]phenyl}-1,3,4-thiadiazol-2-yl)-2,4,6,7-tetrahydro-5H-pyrazolo[4,3-c]pyridin-5-yl]butanoate (Preparation 16) (50 mg, 0.104 mmol) in THF (2 ml) and water (1 ml) was treated with LiOH (5.0 mg, 0.208 mmol) at room temperature and the resulting mixture was stirred at this temperature for 4.5 hours. More LiOH (10.0 mg, 0.416 mmol) was added and the resulting mixture was stirred at the same temperature for 16 hours then was diluted with AcOEt. The organic phase was sequentially washed with a saturated NH₄Cl aqueous solution and water then dried using a phase separator cartridge and concentrated in vacuo to give (8 mg, 17%) as a white solid.

LCMS: Retention time 0.79 min; [M+H]⁺=453

### EXAMPLE 14

### 1-(5-{3-Chloro-4-[(1-methylethyl)oxy]phenyl}-1,3,4-thiadiazol-2-yl)-4,5,6,7-tetrahydro-1H-pyrazolo[4,3-c]pyridine trifluoroacetic acid salt

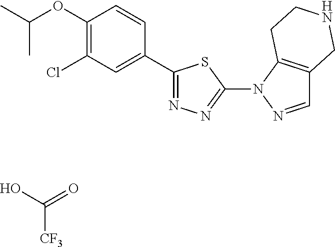

A solution of 1,1-dimethylethyl 1-(5-{3-chloro-4-[(1-methylethyl)oxy]phenyl}-1,3,4-thiadiazol-2-yl)-1,4,6,7-tetrahydro-5H-pyrazolo[4,3-c]pyridine-5-carboxylate (Preparation 12b) (35 mg, 0.074 mmol) in DCM (2 ml) was treated at room temperature with trifluoroacetic acid (0.14 ml, 1.838 mmol) and the resulting mixture was stirred at this temperature for 16 hours. More trifluoroacetic acid (0.14 ml, 1.838 mmol) was added and the resulting mixture was stirred for another 2 hours at room temperature then concentrated in vacuo to give 1-(5-{3-chloro-4-[(1-methylethyl)oxy]phenyl}-1,3,4-thiadiazol-2-yl)-4,5,6,7-tetrahydro-1H-pyrazolo[4,3-c]pyridine trifluoroacetic acid salt (30 mg, 83%) as a pale yellow solid.

LCMS: Retention time 1.06 min; [M+H]⁺=376

### EXAMPLE 15

### 3-[1-(5-{3-Chloro-4-[(1-methylethyl)oxy]phenyl}-1,3,4-thiadiazol-2-yl)-1,4,6,7-tetrahydro-5H-pyrazolo[4,3-c]pyridin-5-yl]propanoic acid

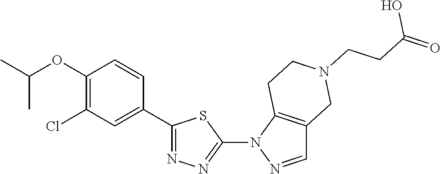

A solution of 1-(5-{3-chloro-4-[(1-methylethyl)oxy]phenyl}-1,3,4-thiadiazol-2-yl)-4,5,6,7-tetrahydro-1H-pyrazolo[4,3-c]pyridine trifluoroacetic acid salt (Example 14) (14 mg, 0.037 mmol), t-butyl acrylate (10.8 μl, 0.074 mmol) and triethylamine (0.02 ml, 0.112 mmol) in n-BuOH (1.5 ml) and THF (0.5 ml) was stirred at 100° C. for 60 minutes under microwave irradiation then cooled to room temperature. More t-butyl acrylate (10.8 μl, 0.074 mmol) was added and the mixture was stirred again at 110° C. for 60 minutes under microwave irradiation then cooled to room temperature and loaded onto a SCX cartridge (5 g) then eluted sequentially with MeOH and 2N NH₃ solution in MeOH. The ammonia fractions were concentrated in vacuo and the residue was dissolved in DCM (1 ml) then treated with trifluoroacetic acid (4.25 mg, 0.037 mmol). The resulting mixture was stirred at room temperature for 16 hours then concentrated in vacuo. Purification of the residue with MDAP (Method Formate) gave 3-[1-(5-{3-chloro-4-[(1-methylethyl)oxy]phenyl}-1,3,4-thiadiazol-2-yl)-1,4,6,7-tetrahydro-5H-pyrazolo[4,3-c]pyridin-5-yl]propanoic acid (6 mg, 36%) as a white oily solid.

LCMS: Retention time 0.99 min; [M+H]⁺=448, 450

### EXAMPLE 16

### 4-[2-(5-{3-Chloro-4-[(1-methylethyl)oxy]phenyl}-1,3,4-thiadiazol-2-yl)-4,5,7,8-tetrahydropyrazolo[3,4-c]azepin-6(2H)-yl]butanoic acid lithium salt

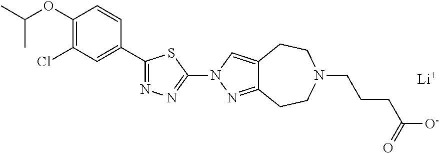

A solution of methyl 4-[2-(5-{3-chloro-4-[(1-methylethyl)oxy]phenyl}-1,3,4-thiadiazol-2-yl)-4,5,7,8-tetrahydropyrazolo[3,4-d]azepin-6(2H)-yl]butanoate (Preparation 20) (23 mg, 0.047 mmol) in THF (0.5 ml) and MeOH (0.500 ml) was treated with a 1N LiOH aqueous solution (0.047 ml, 0.047 mmol) and the resulting mixture was stirred at 65° C. under nitrogen for 3 hours then cooled to room temperature overnight and then concentrated in vacuo. The residue was dissolved in MeOH (2 ml) and the resulting suspension was sonicated. The insoluble material was filtered off and the organic phase was concentrated in vacuo to give 4-[2-(5-{3-chloro-4-[(1-methylethyl)oxy]phenyl}-1,3,4-thiadiazol-2-yl)-4,5,7,8-tetrahydropyrazolo[3,4-d]azepin-6(2H)-yl]butanoic acid lithium salt lithium salt (22 mg, 97%) as a pale yellow solid.

LCMS: Retention time 0.99 min; [M+H]⁺=476, 478

### EXAMPLE 18

### 2-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-N-[2-hydroxy-1-(hydroxymethyl)ethyl]acetamide

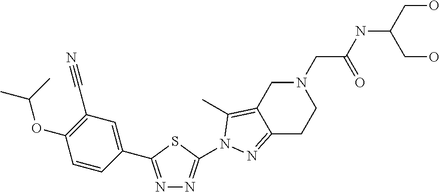

To [2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]acetic acid (Example 39) (95 mg, 0.217 mmol) and HATU (99 mg, 0.260 mmol) in N,N-Dimethylformamide (3 mL) was added DIPEA (0.114 mL, 0.650 mmol) and the reaction mixture stirred at room temperature for 10 mins. 2-amino-1,3-propanediol hydrochloride (27.6 mg, 0.217 mmol) was added and the reaction mixture stirred at room temperature overnight. The sample was partitioned between 1:1 ethyl acetate:dichloromethane (3×10 mL) and water (10 mL). The organic fractions were combined, dried through a hydrophobic frit and concentrated under a stream of nitrogen. The residue was dissolved in DMSO (1 mL) and purified by Mass Directed AutoPrep (Method HpH) The solvent was dried under a stream of nitrogen to give crude product (60 mg). The sample was partitioned between 1:1 ethyl acetate:dichloromethane (2×10 mL) and water (10 mL) to remove excess ammonium chloride. The organics were combined, dried through a hydrophobic frit and evaporated under a stream of nitrogen to give 2-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-N-[2-hydroxy-1-(hydroxymethyl)ethyl]acetamide (32 mg, 29%).

LCMS: Retention time 0.82 min; [M+H]⁺=512

The following compounds were similarly prepared from [2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]acetic acid (Example 39) (95 mg, 0.217 mmol)

| | | | Mass | LCMS |
|---|---|---|---|---|
| | | | mg | Retention |
| | | | (yield | time (min); |
| Example | Amine | Name | %) | [M + H]⁺ |
| 19 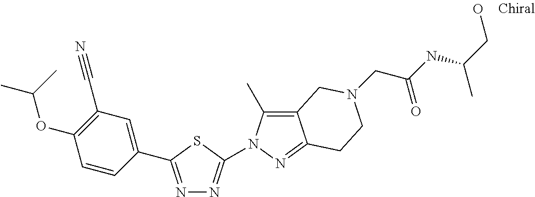 | (2S)-2 amino-1- propanol | 2-[2-(5-{3-cyano-4- [(1-methylethyl)oxy] phenyl}-1,3,4- thiadiazol-2-yl)-3- methyl-2,4,6,7- tetrahydro-5H- pyrazolo[4,3- c]pyridin-5-yl]-N- [(1S)-2-hydroxy-1- methylethyl]acetamide | 58 (54) | 0.85; 496 |
| 20 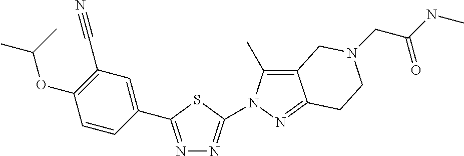 | (2R)-2- amino-1- propanol | 2-[2-(5-{3-cyano-4- [(1-methylethyl)oxy] phenyl}-1,3,4- thiadiazol-2-yl)-3- methyl-2,4,6,7- tetrahydro-5H- pyrazolo[4,3- c]pyridin-5-yl]-N- [(1R)-2-hydroxy-1- methylethyl]acetamide | 81 (75) | 0.85; 496 |
| 21 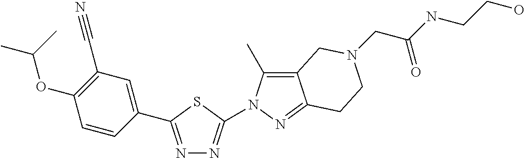 | 2- amino- ethanol | 2-[2-(5-{3-cyano-4- [(1-methylethyl)oxy] phenyl}-1,3,4- thiadiazol-2-yl)-3- methyl-2,4,6,7- tetrahydro-5H- pyrazolo[4,3- c]pyridin-5-yl]-N-(2- hydroxyethyl)acetamide | 53 (51) | 0.83; 482 |
| 22 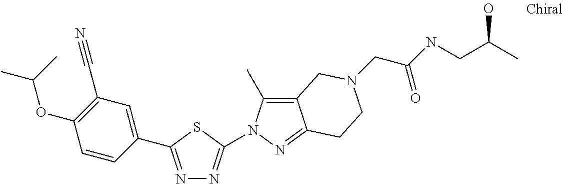 | (2S)-1- amino-2- propanol | 2-[2-(5-{3-cyano-4- [(1-methylethyl)oxy] phenyl}-1,3,4- thiadiazol-2-yl)-3- methyl-2,4,6,7- tetrahydro-5H- pyrazolo[4,3- c]pyridin-5-yl]-N- [(2S)-2- hydroxypropyl] acetamide | 21 (20) | 0.86; 496 |
| 23 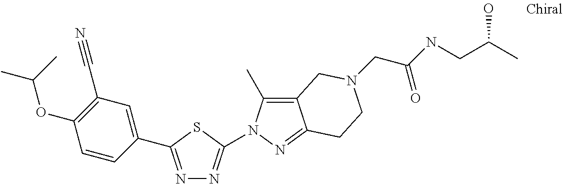 | (2R)-1- amino-2- propanol | 2-[2-(5-{3-cyano-4- [(1-methylethyl)oxy] phenyl}-1,3,4- thiadiazol-2-yl)-3- methyl-2,4,6,7- tetrahydro-5H- pyrazolo[4,3- c]pyridin-5-yl]-N- [(2R)-2- hydroxypropyl] acetamide | 26 (24) | 0.86; 496 |

### EXAMPLE 24a

### 5-{5-[5-(2-hydroxyethyl)-3-methyl-4,5,6,7-tetrahydro-2H-pyrazolo[4,3-c]pyridin-2-yl]-1,3,4-thiadiazol-2-yl}-2-[(1-methylethyl)oxy]benzonitrile

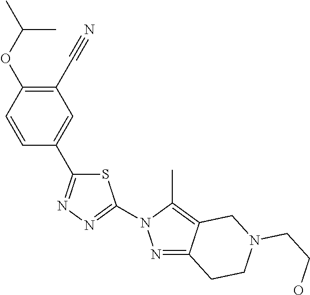

A mixture of 2-[(1-methylethyl)oxy]-5-[5-(3-methyl-4,5,6,7-tetrahydro-2H-pyrazolo[4,3-c]pyridin-2-yl)-1,3,4-thiadiazol-2-yl]benzonitrile (Example 3) (150 mg, 0.3 mmol), 2-bromoethanol (40 mg, 23 μL, 0.32 mmol) and potassium carbonate (126 mg, 0.9 mmol) in acetonitrile (5 mL) was stirred at 50° C. for 24 hours. LCMS showed reaction incomplete but work up anyway. The reaction mixture was cooled and filtered. The solvent was evaporated from the filtrate and the residue chromatographed [0-10% methanol dichloromethane] to give the product as a pale yellow solid (35 mg, 27%)

LCMS: Retention time 0.78 min; [M+H]⁺=425

### EXAMPLE 24b

### 5-{5-[5-(2-hydroxyethyl)-3-methyl-4,5,6,7-tetrahydro-2H-pyrazolo[4,3-c]pyridin-2-yl]-1,3,4-thiadiazol-2-yl}-2-[(1-methylethyl)oxy]benzonitrile hydrochloride

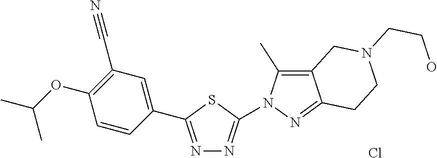

To a stirred solution of 5-{5-[5-(2-hydroxyethyl)-3-methyl-4,5,6,7-tetrahydro-2H-pyrazolo[4,3-c]pyridin-2-yl]-1,3,4-thiadiazol-2-yl}-2-[(1-methylethyl)oxy]benzonitrile (Example 24a) (102 mg, 0.24 mmol) in Methanol (5 mL) was added 1M hydrogen chloride in ether (1.2 mL, 1.2 mmol). The mixture was allowed to stir for 5 min at room temperature before being evaporated under a stream of nitrogen and then dried in vacuo to give 5-{5-[5-(2-hydroxyethyl)-3-methyl-4,5,6,7-tetrahydro-2H-pyrazolo[4,3-c]pyridin-2-yl]-1,3,4-thiadiazol-2-yl}-2-[(1-methylethyl)oxy]benzonitrile hydrochloride as a cream solid (100 mg, 90%)

LCMS: Retention time 0.81 min; [M+H]⁺=425

### EXAMPLE 25a

### 5-(5-{5-[2-hydroxy-1-(hydroxymethyl)ethyl]-3-methyl-4,5,6,7-tetrahydro-2H-pyrazolo[4,3-c]pyridin-2-yl}-1,3,4-thiadiazol-2-yl)-2-[(1-methylethyl)oxy]benzonitrile

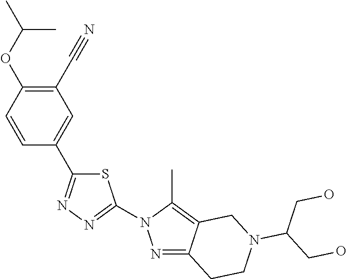

2M Hydrochloric acid (3 mL) was added to a stirred solution of 5-{5-[5-(2,2-dimethyl-1,3-dioxan-5-yl)-3-methyl-4,5,6,7-tetrahydro-2H-pyrazolo[4,3-c]pyridin-2-yl]-1,3,4-thiadiazol-2-yl}-2-[(1-methylethyl)oxy]benzonitrile (Preparation 21) (245 mg, 0.5 mmol) in THF (3 mL). The reaction mixture was stirred at room temperature for 24 hours. The reaction was quenched with saturated NaHCO₃ (10 mL) and extracted with ethyl acetate (3×10 mL). The combined extracts were washed with water and brine. Dried and evaporated. The residue was chromatographed [5-10% methanol/dichloromethane] to give 5-(5-{5-[2-hydroxy-1-(hydroxymethyl)ethyl]-3-methyl-4,5,6,7-tetrahydro-2H-pyrazolo[4,3-c]pyridin-2-yl}-1,3,4-thiadiazol-2-yl)-2-[(1-methylethyl)oxy]benzonitrile (95 mg, 42%)

LCMS: Retention time 0.79 min; [M+H]⁺=455

### EXAMPLE 25b

### 5-(5-{5-[2-hydroxy-1-(hydroxymethyl)ethyl]-3-methyl-4,5,6,7-tetrahydro-2H-pyrazolo[4,3-c]pyridin-2-yl}-1,3,4-thiadiazol-2-yl)-2-[(1-methylethyl)oxy]benzonitrile hydrochloride

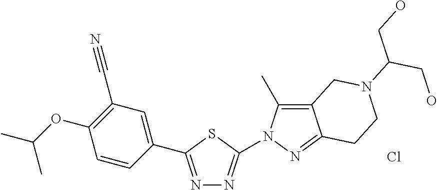

To a stirred solution of 5-(5-{5-[2-hydroxy-1-(hydroxymethyl)ethyl]-3-methyl-4,5,6,7-tetrahydro-2H-pyrazolo[4,3-c]pyridin-2-yl}-1,3,4-thiadiazol-2-yl)-2-[(1-methylethyl)oxy]benzonitrile (Example 25a) (6.0 mg, 0.013 mmol) in Methanol (2 mL) was added 1M hydrogen chloride in ether (0.10 mL, 0.100 mmol). The mixture was allowed to stir for 5 min at room temperature before being evaporated under a stream of nitrogen and then dried in vacuo to give 5-(5-{5-[2-hydroxy-1-(hydroxymethyl)ethyl]-3-methyl-4,5,6,7-tetrahydro-2H-pyrazolo[4,3-c]pyridin-2-yl}-1,3,4-thiadiazol-2-yl)-2-[(1-methylethyl)oxy]benzonitrile hydrochloride (5.3 mg, 82%)

LCMS: Retention time 0.82 min; [M+H]⁺=455

### EXAMPLE 26

### 5-(5-{5-[(2S)-2,3-dihydroxypropyl]-3-methyl-4,5,6,7-tetrahydro-2H-pyrazolo[4,3-c]pyridin-2-yl}-1,3,4-thiadiazol-2-yl)-2-[(1-methylethyl)oxy]benzonitrile

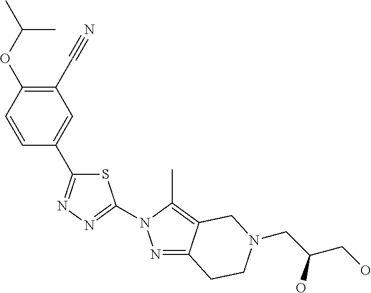

D-gylceraldehyde (91 mg, 1.01 mmol) was added to a stirred solution of 2-[(1-methylethyl)oxy]-5-[5-(3-methyl-4,5,6,7-tetrahydro-2H-pyrazolo[4,3-c]pyridin-2-yl)-1,3,4-thiadiazol-2-yl]benzonitrile (Example 3) (100 mg, 0.20 mmol) in 1,2-dichloroethane (2 mL), THF (2 mL) and methanol (1 mL). The reaction mixture was stirred at room temperature for 30 minutes then treated with sodium triacetoxyborohydride (214 mg, 1.01 mmol). The reaction mixture was stirred at room temperature overnight. Saturated NaHCO₃ (10 mL) was added and the mixture extracted with ethyl acetate (3×10 mL). The combined extracts were washed with water and brine. Dried and evaporated. The residue was chromatographed [0-5% methanol/dichloromethane] to give the product. Trituration with diethyl ether gave a colourless solid (49 mg, 53%)

LCMS: Retention time 0.77 min; [M+H]⁺=455

### EXAMPLE 27

### 5-(5-{5-[(2R)-2,3-dihydroxypropyl]-3-methyl-4,5,6,7-tetrahydro-2H-pyrazolo[4,3-c]pyridin-2-yl}-1,3,4-thiadiazol-2-yl)-2-[(1-methylethyl)oxy]benzonitrile

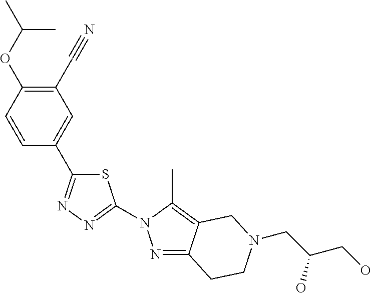

L-Glyceraldehyde (182 mg, 2.02 mmol) as added to a stirred solution of 2-[(1-methylethyl)oxy]-5-[5-(3-methyl-4,5,6,7-tetrahydro-2H-pyrazolo[4,3-c]pyridin-2-yl)-1,3,4-thiadiazol-2-yl]benzonitrile (Example 3) (200 mg, 0.4 mmol) in dichloromethane (5 mL) and methanol (1 mL). After stirring for 15 minutes sodium triacetoxyborohydride (429 mg, 2.02 mmol) was added and stirring continued overnight at room temperature. Saturated NaHCO₃ (10 mL) was added and the mixture extracted with ethyl acetate (3×10 mL). The combined extracts were washed with brine, dried and evaporated. The residue was chromatographed [2-10% methanol/dichloromethane] twice and pure fractions combined to give 5-(5-{5-[(2R)-2,3-dihydroxypropyl]-3-methyl-4,5,6,7-tetrahydro-2H-pyrazolo[4,3-c]pyridin-2-yl}-1,3,4-thiadiazol-2-yl)-2-[(1-methylethyl)oxy]benzonitrile as a colourless solid (20 mg, 11%)

LCMS: Retention time 0.77 min; [M+H]⁺=455

### EXAMPLE 28

### 5-{5-[5-(3-hydroxypropyl)-3-methyl-4,5,6,7-tetrahydro-2H-pyrazolo[4,3-c]pyridin-2-yl]-1,3,4-thiadiazol-2-yl}-2-[(1-methylethyl)oxy]benzonitrile

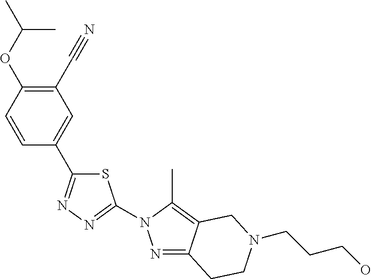

A mixture of 2-[(1-methylethyl)oxy]-5-[5-(3-methyl-4,5,6,7-tetrahydro-2H-pyrazolo[4,3-c]pyridin-2-yl)-1,3,4-thiadiazol-2-yl]benzonitrile (Example 3)(150 mg, 0.3 mmol), 3-bromo-1-propanol (45 mg, 29 μL, 0.32 mmol) and potassium carbonate (126 mg, 0.9 mmol) in acetonitrile (5 mL) was stirred at 50° C. for 24 hours. The reaction mixture was cooled and filtered. The solvent was evaporated from the filtrate and the residue chromatographed [0-10% methanol dichloromethane] to give the product as a pale yellow solid (44 mg, 33%)

LCMS: Retention time 0.79 min; [M+H]⁺=439

### EXAMPLE 29

### 5-[5-(5-glycyl-3-methyl-4,5,6,7-tetrahydro-2H-pyrazolo[4,3-c]pyridin-2-yl)-1,3,4-thiadiazol-2-yl]-2-[(1-methylethyl)oxy]benzonitrile

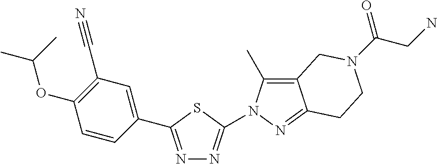

To 1,1-dimethylethyl {2-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-2-oxoethyl}carbamate (Preparation 22) (88 mg, 0.16 mmol) in dichloromethane (2 mL) was added trifluoroacetic acid (0.28 mL, 3.68 mmol) and the reaction mixture stirred at room temperature for 1 hour. The reaction mixture was applied directly to an aminopropyl SPE cartridge (2 g) and eluted using 10% methanol in dichloromethane. The appropriate fractions were combined and evaporated. The residue was dissolved in DMSO (1 mL) and purified by MDAP (Method HpH). The fraction was then collected and partitioned between sodium bicarbonate solution and ethyl acetate. The organic layer was separated and filtered through a hydrophobic frit. The solvent was evaporated to give 5-[5-(5-glycyl-3-methyl-4,5,6,7-tetrahydro-2H-pyrazolo[4,3-c]pyridin-2-yl)-1,3,4-thiadiazol-2-yl]-2-[(1-methylethyl)oxy]benzonitrile (25 mg, 33% yield).

LCMS: Retention time 0.87 min; [M+H]⁺=438

### EXAMPLE 30

### 5-[5-(5-{N—[(1R)-2-hydroxy-1-methylethyl]glycyl}-3-methyl-4,5,6,7-tetrahydro-2H-pyrazolo[4,3-c]pyridin-2-yl)-1,3,4-thiadiazol-2-yl]-2-[(1-methylethyl)oxy]benzonitrile

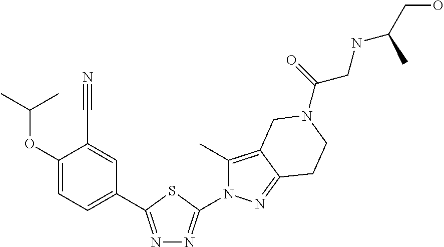

5-{5-[5-(bromoacetyl)-3-methyl-4,5,6,7-tetrahydro-2H-pyrazolo[4,3-c]pyridin-2-yl]-1,3,4-thiadiazol-2-yl}-2-[(1-methylethyl)oxy]benzonitrile (Preparation 22) (75 mg, 0.150 mmol), potassium iodide (3 mg, 0.018 mmol) and potassium carbonate (51.7 mg, 0.374 mmol) were dissolved in Acetonitrile (2 mL) at room temperature. (2R)-2-amino-1-propanol (0.058 mL, 0.75 mmol) was added and stirred for 16 hours. The mixture was partitioned between DCM and water and the organic extracted and the aqueous washed with DCM. The combined organic extracts were dried with a hydrophobic frit and evaporated to give a residue which was purified by MDAP (Method HpH). This solution was blown down and converted to the salt by dissolving the residue in methanol (2 mL) and adding hydrochloric acid in ether (1M, 200 μL). Sample was converted back to free base by dissolving in methanol (2 mL) and adding ammonia (0.88M, 100 μL) This gave the product 5-[5-(5-{N—[(1R)-2-hydroxy-1-methylethyl]glycyl}-3-methyl-4,5,6,7-tetrahydro-2H-pyrazolo[4,3-c]pyridin-2-yl)-1,3,4-thiadiazol-2-yl]-2-[(1-methylethyl)oxy]benzonitrile (25 mg, 32% yield)

LCMS: Retention time 0.92 min; [M+H]⁺=496

The following compounds were prepared in an analogous fashion starting with 5-{5-[5-(bromoacetyl)-3-methyl-4,5,6,7-tetrahydro-2H-pyrazolo[4,3-c]pyridin-2-yl]-1,3,4-thiadiazol-2-yl}-2-[(1-methylethyl)oxy]benzonitrile (Preparation 22) (75 mg, 0.150 mmol) and the appropriate amine;

| | | Mass | LCMS retention |
|---|---|---|---|
| | | mg | time (min); |
| Example | Name | (yield %) | [M+ H]⁺ |
| 31 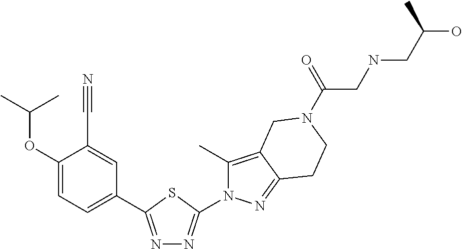 | 5-[5-(5-{N-[(1S)-2- hydroxy-1- methylethyl]glycyl}-3- methyl-4,5,6,7- tetrahydro-2H- pyrazolo[4,3-c]pyridin-2- yl)-1,3,4-thiadiazol-2-yl]- 2-[(1-methylethyl)oxy] benzonitrile | 26 (33) | 0.90; 496 |
| 32 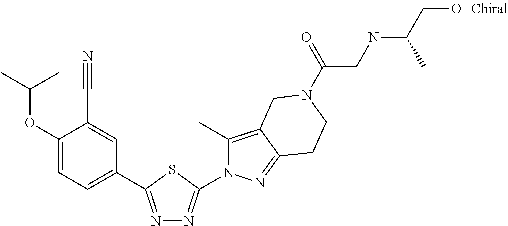 | 5-[5-(5-{N-[(2R)-2- hydroxypropyl]glycyl}-3- methyl-4,5,6,7- tetrahydro-2H- pyrazolo[4,3-c]pyridin-2- yl)-1,3,4-thiadiazol-2-yl]- 2-[(1-methylethyl)oxy] benzonitrile | 7 (9) | 0.92; 496 |
| 33 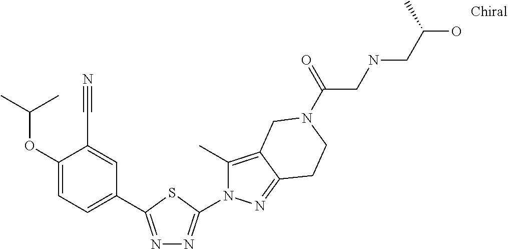 | 5-[5-(5-{N-[(2S)-2- hydroxypropyl]glycyl}-3- methyl-4,5,6,7- tetrahydro-2H- pyrazolo[4,3-c]pyridin-2- yl)-1,3,4-thiadiazol-2-yl]- 2-[(1-methylethyl)oxy] benzonitrile GSK2413002A | 12 (15) | 0.92; 496 |
| 34 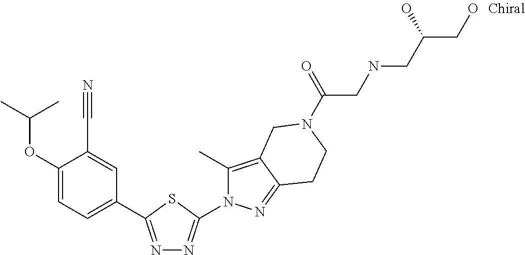 | 5-[5-(5-{N-[(2S)-2,3- dihydroxypropyl]glycyl}- 3-methyl-4,5,6,7- tetrahydro-2H- pyrazolo[4,3-c]pyridin-2- yl)-1,3,4-thiadiazol-2-yl]- 2-[(1-methylethyl)oxy] benzonitrile | 2 (2) | 0.89; 512 |
| 35 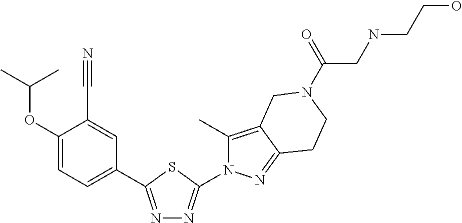 | 5-(5-{5-[N-(2- hydroxyethyl)glycyl]-3- methyl-4,5,6,7- tetrahydro-2H- pyrazolo[4,3-c]pyridin-2- yl}-1,3,4-thiadiazol-2-yl)- 2-[(1-methylethyl)oxy] benzonitrile | 8 (10) | 0.90; 482 |
| 36 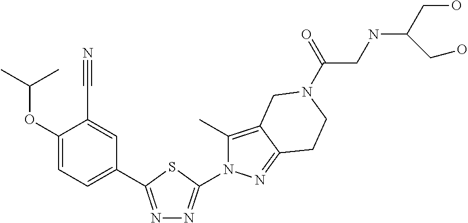 | 5-[5-(5-{N-[2-hydroxy-1- (hydroxymethyl)ethyl] glycyl}-3-methyl-4,5,6,7- tetrahydro-2H- pyrazolo[4,3-c]pyridin-2- yl)-1,3,4-thiadiazol-2-yl]- 2-[(1-methylethyl)oxy] benzonitrile | 11 (13) | 0.90; 512 |
| 37 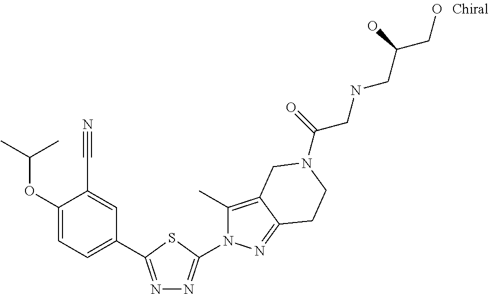 | 5-[5-(5-{N-[(2R)-2,3- dihydroxypropyl]glycyl}- 3-methyl-4,5,6,7- tetrahydro-2H- pyrazolo[4,3-c]pyridin-2- yl)-1,3,4-thiadiazol-2-yl]- 2-[(1-methylethyl)oxy] benzonitrile | 2 (2) | 0.91; 512 |

### EXAMPLE 38

### [2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]acetic acid

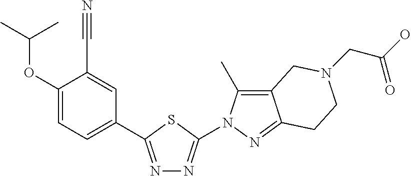

Trifluoroacetic acid (3 mL) was added slowly to a stirred solution of 1,1-dimethylethyl [2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]acetate (Preparation 23) (470 mg, 0.95 mmol) in dichloromethane (10 mL). The reaction mixture was stirred at room temperature for 4 hours. The solvent was evaporated and the residue re-evaporated from toluene (×2). The residue was triturated with diethyl ether to give [2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]acetic acid as an off-white solid (467 mg, 89%)

LCMS: Retention time 0.81 min; [M+H]⁺=439

### EXAMPLE 39a

### 2-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-1,3-propanediol

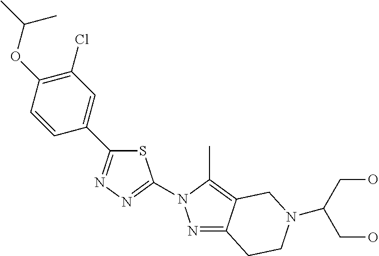

2-(5-{3-chloro-4-[(1-methylethyl)oxy]phenyl}-1,3,4-thiadiazol-2-yl)-3-methyl-4,5,6,7-tetrahydro-2H-pyrazolo[4,3-c]pyridine (Example 1)(150 mg, 0.39 mmol), 2,2-dimethyl-1,3-dioxan-5-one (0.184 mL, 1.54 mmol) and sodium triacetoxyborohydride (326 mg, 1.54 mmol) were dissolved in dichloromethane (DCM) (5 mL) and stirred at RT under N₂ for 2 days. The mixture was diluted with DCM (10 mL) and washed with saturated aqueous sodium bicarbonate solution, then the mixture was separated on a phase separation cartridge and the chlorinated phase evaporated under vacuum to give a dark brown oil. The oil was loaded onto an SCX cartridge (10 g) and washed with methanol (2×20 mL) the product was eluted with ammonia in methanol (2M, 2×20 mL) and solvent evaporated under vacuum. Wash through SCX repeated using the same process. Residue triturated with DCM and filtered to give solid 2-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-1,3-propanediol (65 mg, 36%)

LCMS: Retention time 0.87 min; [M+H]⁺=464

### EXAMPLE 39b

### 2-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-1,3-propanediol hydrochloride

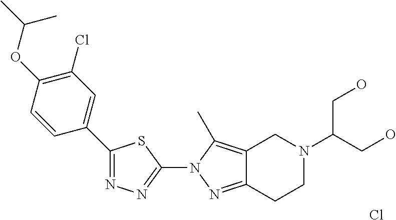

To 2-(5-{3-chloro-4-[(1-methylethyl)oxy]phenyl}-1,3,4-thiadiazol-2-yl)-5-(2,2-dimethyl-1,3-dioxan-5-yl)-3-methyl-4,5,6,7-tetrahydro-2H-pyrazolo[4,3-c]pyridine (Preparation 24) (374 mg, 0.742 mmol) in Tetrahydrofuran (THF) (6 mL) was added 2M aqueous hydrochloric acid (6.00 mL, 197 mmol) and the reaction mixture stirred at room temperature for 2 days. The reaction mixture was concentrated under reduced pressure to get a yellow solid (360 mg, 92%)

LCMS: Retention time 0.90 min; [M+H]⁺=464, 466

1H NMR (400 MHz, DMSO-d₆) d (ppm): 10.49 (br. s., 1H), 8.04 (d, J=2.0 Hz, 1H), 7.90 (dd, J=9.0, 2.0 Hz, 1H), 7.36 (d, J=9.0 Hz, 1H), 4.83 (spt, J=6.0 Hz, 1H), 4.50-4.54 (m, 2H), 3.83-3.96 (m, 5H), 3.50-3.65 (m, 1H), 3.40-3.48 (m, 1H), 3.15-3.27 (m, 1H), 2.99-3.10 (m, 1H), 2.66 (s, 3H), 1.34 (d, J=6.0 Hz, 6H)

### EXAMPLE 40

### (2R)-3-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-1,2-propanediol formic acid salt

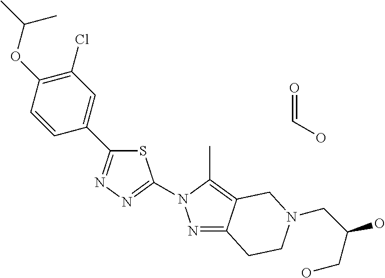

2-(5-{3-chloro-4-[(1-methylethyl)oxy]phenyl}-1,3,4-thiadiazol-2-yl)-3-methyl-4,5,6,7-tetrahydro-2H-pyrazolo[4,3-c]pyridine (Example 1)(150 mg, 0.39 mmol), (2S)-2,3-dihydroxypropanal (139 mg, 1.54 mmol) and sodium triacetoxyborohydride (326 mg, 1.54 mmol) were dissolved in dichloromethane (DCM) (4.5 mL) and Methanol (0.5 mL) at RT under N₂ and stirred for 2 days. More aldehyde (70 mg, 2 eq.) and sodium triacetoxyborohydride (163 mg, 2 eq.) were added with more MeOH (1 mL) and DCM (1 mL) and stirring continued for 3 days. The mixture was diluted with DCM (10 mL) and washed with saturated aqueous sodium bicarbonate solution, then the mixture was separated on a phase sep cartridge and the chlorinated phase and evaporated under vacuum to give a dark brown oil which was dissolved in 1:1 MeOH:DMSO (1 mL) and purified by Mass Directed AutoPrep (Method Formate). The solvent was evaporated under vacuum to give an off white solid (2R)-3-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-1,2-propanediol formic acid salt (97 mg, 49.4%)

LCMS: Retention time 0.82 min; [M+H]⁺=464

### EXAMPLE 41

### methyl (2R)-3-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-2-hydroxypropanoate

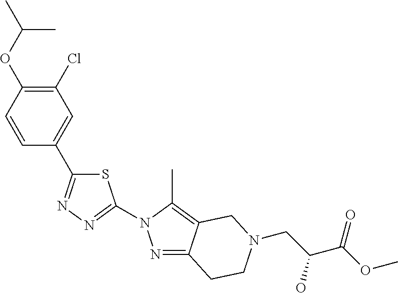

2-(5-{3-chloro-4-[(1-methylethyl)oxy]phenyl}-1,3,4-thiadiazol-2-yl)-3-methyl-4,5,6,7-tetrahydro-2H-pyrazolo[4,3-c]pyridine (Example 1)(148 mg, 0.38 mmol), methyl (2R)-2-oxiranecarboxylate (0.040 mL, 0.455 mmol) and DIPEA (0.199 mL, 1.14 mmol) were dissolved in N,N-Dimethylformamide (DMF) (1 mL) and heated at 160° C. in the microwave for 30 mins. The reaction allowed to cool overnight then quenched with saturated aqueous sodium bicarbonate solution and extracted into DCM (20 mL). The mixture was separated on a phase separation cartridge and the chlorinated phase evaporated under vacuum to give a brown residue. The residue was purified by Biotage SP4 silica gel SNAP (50 g) column using a gradient of 0.5-5% dichloromethane-methanol. The appropriate fractions were combined and evaporated under vacuum to give an off white glassy solid methyl (2R)-3-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-2-hydroxypropanoate (144 mg, 77%).

LCMS: Retention time 0.93 min; [M+H]⁺=492

### EXAMPLE 42

### (2S)-3-[2-(5-{3-chloro-4-[(1-methylethyl)oxy]phenyl}-1,3,4-thiadiazol-2-yl)-3-methyl-2,4,6,7-tetrahydro-5H-pyrazol o[4,3-c]pyridin-5-yl]-1,2-propanediol formic acid salt

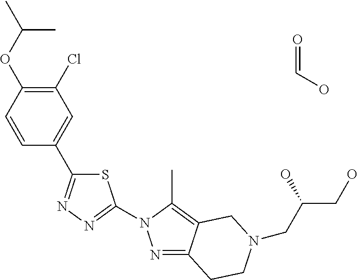

2-(5-{3-chloro-4-[(1-methylethyl)oxy]phenyl}-1,3,4-thiadiazol-2-yl)-3-methyl-4,5,6,7-tetrahydro-2H-pyrazolo[4,3-c]pyridine (Example 1) (150 mg, 0.39 mmol), (2R)-2,3-dihydroxypropanal (139 mg, 1.54 mmol) and sodium triacetoxyborohydride (326 mg, 1.539 mmol) were dissolved in dichloromethane (4.5 mL) and methanol (0.5 mL) at RT under N₂ and stirred for 2 days. More (2R)-2,3-dihydroxypropanal (70 mg, 2 eq.) and sodium triacetoxyborohydride (163 mg, 2 eq.) were added with more MeOH (1 mL) and DCM (1 mL) and stirring continued for 3 days. The mixture was diluted with DCM (10 mL) and washed with saturated aqueous sodium bicarbonate solution, then the mixture was separated on a phase separation cartridge and the chlorinated phase and evaporated under vacuum to give a dark brown oil which was dissolved in 1:1 MeOH:DMSO (1 mL) and purified by Mass Directed AutoPrep (Method Formate). The solvent was evaporated under vacuum to give an off white solid. Product fractions were combined and evaporated to give a yellow solid (2S)-3-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-1,2-propanediol formic acid salt (19 mg, 10%)

LCMS: Retention time 0.91 min; [M+H]⁺=464

### EXAMPLE 43

### 2-[(1-methylethyl)oxy]-5-[5-(3-methyl-4,5,6,7-tetrahydro-2H-pyrazolo[3,4-c]pyridin-2-yl)-1,3,4-thiadiazol-2-yl]benzonitrile hydrochloride

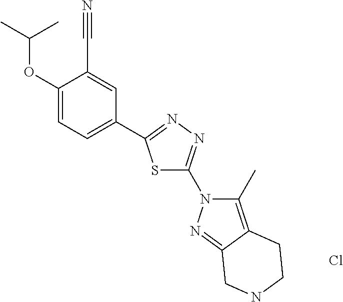

A mixture of 1,1-dimethylethyl 2-(5-{3-cyano-4-[(1-methylethyl)oxy]phenyl}-1,3,4-thiadiazol-2-yl)-3-methyl-2,4,5,7-tetrahydro-6H-pyrazolo[3,4-c]pyridine-6-carboxylate (Preparation 25) (10 mg, 0.02 mmol), dioxan (0.5 mL) and hydrogen chloride in dioxan (4M, 0.5 mL) was stirred at room temperature overnight. Diethyl ether (5 mL) was added. The precipitate was filtered off, washed with diethyl ether and dried to give an off-white solid 2-[(1-methylethyl)oxy]-5-[5-(3-methyl-4,5,6,7-tetrahydro-2H-pyrazolo[3,4-c]pyridin-2-yl)-1,3,4-thiadiazol-2-yl]benzonitrile hydrochloride (8 mg, 92%)

LCMS: Retention time 0.83 min; [M+H]⁺=381

### EXAMPLE 44

### 2-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-N-(2-hydroxy-1,1-dimethylethyl)acetamide

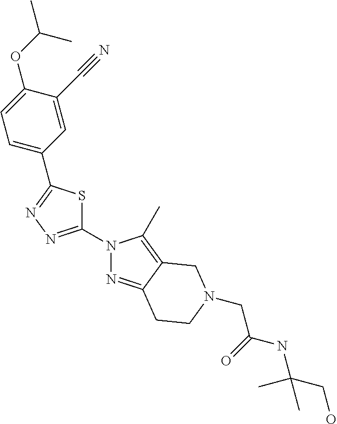

2-Amino-2-methylpropan-1-ol (65 mg, 0.72 mmol) was added to a stirred mixture of [2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]acetic acid (Example 38) (200 mg, 0.36 mmol), HATU (165 mg, 0.43 mmol), and N-ethylmorpholine (83 mg, 0.72 mmol) in DMF (5 mL). The reaction mixture was stirred at room temperature for 48 hours.

Saturated NaHCO₃ (15 mL) was added and then the mixture was extracted with ethyl acetate (2×20 mL). The combined extracts were washed with water and brine, dried and evaporated. The residue was chromatographed [0-4% methanol/dichloromethane] to give the product as a pale yellow solid (120 mg, 65%)

LCMS: Retention time 0.83 min; [M+H]⁺=510

### Membrane Preparation for S1P1 GTPγS Assay

All steps were performed at 4° C. Cells were homogenised within a glass Waring blender for 2 bursts of 15 secs in 200 mls of buffer (50 mM HEPES, 1 mM leupeptin, 25 m/ml bacitracin, 1 mM EDTA, 1 mM PMSF, 2 μM pepstatin A). The blender was plunged into ice for 5 mins after the first burst and 10-40 mins after the final burst to allow foam to dissipate. The material was then spun at 500 g for 20 mins and the supernatant spun for 36 mins at 48,000 g. The pellet was resuspended in the same buffer as above but without PMSF and pepstatin A. The material was then forced through a 0.6 mm needle, made up to the required volume, (usually ×4 the volume of the original cell pellet), aliquoted and stored frozen at—80° C.

### S1P1 GTPγS Assay

S₁P₁ expressing RH7777 membranes (1.5 μg/well) membranes (1.5 μg/well) were homogenised by passing through a 23G needle. These were then adhered to WGA-coated SPA beads (0.125 mg/well) in assay buffer (HEPES 20 mM, MgCl₂, 10 mM, NaCl 100 mM and pH adjusted to 7.4 using KOH 5M). GDP 10 μM FAC and saponin 90 μg/ml FAC were also added

After 30 minutes precoupling on ice, the bead and membrane suspension was dispensed into white Greiner polypropylene LV 384-well plates (5 μl/well), containing 0.1 μl of compound. 5 μl/well [³⁵S]-GTPγS (0.5 nM for S₁P₁ or 0.3 nM for S₁P₃ final radioligand concentration) made in assay buffer was then added to the plates. The final assay cocktail (10.1 μl) was then sealed, spun on a centrifuge, then read immediately on a Viewlux instrument.

Examples 1 to 16 Had a pEC50 of >6 in this Assay.

Alternatively, after 30 minutes precoupling on ice, the bead and membrane suspension was mixed with ³⁵S]-GTPγS (0.5 nM final radioligand concentration) in assay buffer (HEPES 20 mM, MgCl₂ 10 mM, NaCl 100 mM and pH adjusted to 7.4 using KOH 5M) in a 1:1 ratio. The bead, membrane and radioligand suspension was dispensed into white Greiner polypropylene low volume 384-well plates (10 μl/well), containing 0.1 μl of a solution of test compound in 100% DMSO. The final assay cocktail (10.1 μl) was then sealed, spun on a centrifuge, then read immediately on a Viewlux instrument.

Tested in one of the above S1P1 assays Examples 1 to 12, 14 to 16 and 25 to 27 had a pEC50 of >6. Examples 3 to 5, 7, 8, 12 and 25 to 26 had a pEC50 of 7. Example 2 had a pEC50 of 8.

S1P3 GTPγS Assay

S₁P₃ expressing RBL membranes (1.5 μg/well) were homogenised by passing through a 23G needle. These were then adhered to WGA-coated SPA beads (0.125 mg/well) in assay buffer (HEPES 20 mM, MgCl₂ 10 mM, NaCl 100 mM and pH adjusted to 7.4 using KOH 5M). GDP 10 μM FAC and saponin 90 m/ml FAC were also added

After 30 minutes precoupling on ice, the bead and membrane suspension was dispensed into white Greiner polypropylene LV 384-well plates (5 μl/well), containing 0.1 μlof compound. 5 μl/well [³⁵S]-GTPγS (0.5 nM for S₁P₁ or 0.3 nM for S₁P₃ final radioligand concentration) made in assay buffer was then added to the plates. The final assay cocktail (10.1 μl) was then sealed, spun on a centrifuge, then read immediately on a Viewlux instrument.

Examples 1 to 16 had a pEC50 of <6 in this assay.

Alternative membrane preparation for S1P3 GTPγS assay All steps were performed at 4° C. Cells were homogenised within a glass Waring blender for 2 bursts of 15 secs in 200 mls of buffer (50 mM HEPES, 1 mM leupeptin, 25 μg/ml bacitracin, 1 mM EDTA, 1 mM PMSF, 2 μM pepstatin A). The blender was plunged into ice for 5 mins after the first burst and 10-40 mins after the final burst to allow foam to dissipate. The material was then spun at 500 g for 20 mins and the supernatant spun for 36 mins at 48,000 g. The resultant pellet was resuspended in the same buffer without PMSF and pepstatin A but containing 10% w/v sucrose. The membrane suspension was then layered on top of buffer without PMSF and pepstatin A but containing 40% w/v sucrose and spun at 100,000 g for 60 mins. The cloudy interface between the 2 sucrose layers was removed and resuspended in buffer without PMSF and pepstatin A. The material was spun at 48,000 g for 45 mins.

The resultant cell pellet was resuspended in the required volume in buffer without PMSF and pepstatin A, (usually ×4 the volume of the original cell pellet), aliquoted and stored frozen at −80° C.

Alternative S1P3 Purified Membrane GTPγS Assay

S₁P₃ expressing RBL membranes (0.44 μg/well) purified through a sucrose gradient were homogenised by passing through a 23 G needle. These were then adhered to WGA-coated SPA beads (GE Healthcare 0.5 mg/well) in assay buffer (HEPES 20 mM, MgCl₂ 10 mM, NaCl 100 mM and pH adjusted to 7.4 using KOH 5M). 2 m/well of Saponin was added.

After 30 minutes precoupling on ice, 5 μM GDP final assay concentration was added to the bead and membrane suspension. The bead, membrane, Saponin and GDP suspension was mixed with [³⁵5]-GTPγS (Perkin Elmer, 0.3 nM final radioligand concentration) made in assay buffer (HEPES 20 mM, MgCl₂ 10 mM, NaCl 100 mM and pH adjusted to 7.4 using KOH 5M). The bead, membrane and radioligand suspension was dispensed into white Greiner polypropylene 384-well plates (45 μl/well), containing 0.5 μl of a solution of test compound in 100% DMSO. The final assay cocktail (45.5 μl) was then sealed, spun on a centrifuge, then read on a Viewlux instrument following a 3 hour incubation of plates at room temperature.

Tested in one of the above S1P3 assays examples 1 to 15 and 18 to 44 had a pEC50 of <6. Examples 1, 3 to 8, 9 to 15 and 18 to 44 had a pDC50 of <5.

S1P-1 β-Arrestin Recruitment Assay

β-Arrestin recruitment assays were carried out using the PathHunter CHO-K1 EDG1 β-Arrestin cell line (DiscoveRx Corporation) in a chemi-luminescence detection assay. This cell line stably expresses β-Arrestin 2 and S1P1 fused to complementing portions of β-galactosidase (‘EA’ and ‘pro-link’, respectively) which associate upon Arrestin recruitment to form functional β-galactosidase enzyme.

Cells were grown to 80% confluency in Growth Medium (F12 nutrient HAMS supplemented with 10% heat-inactivated USA FBS, 1% L-glutamax, 800 m/ml Geneticin and 300 m/ml Hygromycin). Cells were harvested from the flask using Enzyme Free Cell Dissociation Buffer (Gibco) and washed from flasks with Optimem solution (Gibco). Cells were then centrifuged at 1000 rpm for 2-3 min and resuspended in Assay Buffer (Prepared from Sigma kit H1387 supplemented with 20 ml/L HEPES, 4.7 ml/L NaHCO3, 0.1% pluronic acid F-68 solution, 0.1% BSA and adjusted to pH 7.4 using sodium hydroxide at 1×10⁶ cells/ml. Cells were dispensed into assay plates containing compounds (100n1/well of a solution of test compound in 100% DMSO) at 1×104 cells/well and incubated at 37° C./5% CO2 for 90 min followed by 15 min at room temperature. 5 μl detection mix (1 part Galacton Star, 5 parts Emerald II, 19 parts Assay Buffer; DiscoveRx) were added per well and the plates incubated at room temperature for 60 min. Luminescence was quantified using a Viewlux plate reader. Examples 1 to 16 and 18 to 44 had a pEC50≧6. Examples 3, 15, 18 to 24, 27 to 19, 31, 33, 37 to 40 and 42 had a pEC50 of 7. Examples 2, 4, 5, 7, 8, 12, 13, 25, 26, 30, 32, 34 to 36 and 41 had a pEC50 of ≧8.

1. A compound of formula (I) or a pharmaceutically acceptable salt thereof: 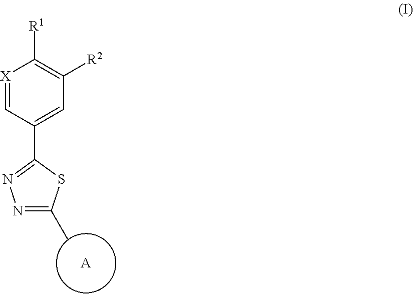
X is CH or N;
R¹ is OR³, NHR⁴, R⁵, NR⁶R⁷, R⁸ or optionally fluorinated C₍₃₋₆₎cycloalkyl;
R² is hydrogen, halogen, cyano, trifluoromethyl, C₍₁₋₂₎alkoxy and C₍₁₋₃₎alkyl;
R³ and R⁴ are C₍₁₋₅₎ alkyl optionally interrupted by O and optionally substituted by F or (CH₂)₍₀₋₁₎C₍₃₋₅₎ cycloalkyl optionally substituted by F;
R⁵ is C₍₁₋₆₎ alkyl optionally substituted by F;
R⁶ and R⁷ are independently selected from C₍₁₋₅₎ alkyl optionally interrupted by O and optionally substituted by F and optionally fluorinated C₍₃₋₅₎cycloalkyl with the proviso that the combined number of carbon atoms in R⁶ and R⁷ does not exceed 6;
R⁸ is a 3 to 6 membered, nitrogen-containing heterocyclyl ring optionally substituted by F selected from aziridinyl, azetidinyl, pyrrolidinyl, piperidinyl and morpholinyl, all attached via the nitrogen atom;
A is a bicyclic ring selected from the following: 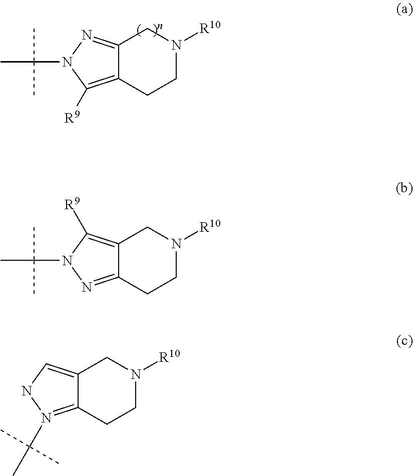
R⁹ is hydrogen or C₍₁₋₃₎alkyl;
R¹⁰ is hydrogen, C₍₁₋₄₎ alkyl, C₍₁₋₄₎alkylCOOH, C₍₁₋₄₎alkylCONR¹¹R¹², C₍₂₋₄₎alkylNR¹³CONR¹¹R¹², C₍₂₋₄₎alkylNR¹³COOR¹², C₍₂₋₄₎alkylOCONR¹¹R¹²C₍₂₋₄₎alkylNR¹³COR¹² or COC₍₁₋₄₎NR¹¹R¹²;
when R¹⁰ comprises an alkyl chain of at least two carbon atoms at the point of attachment to the A ring it may be optionally substituted by halogen, SO₂C₍₁₋₃₎alkyl, or by at least one OH;
R¹¹, R¹² and R¹³ are independently selected from hydrogen or C₍₁₋₃₎alkyl optionally substituted by F or hydroxyl and optionally interrupted by O;
R¹¹ and R¹² together with the nitrogen atom to which they are attached may be linked to form a 4-6 membered heterocyclyl ring, wherein the 4- to 6-membered heterocyclyl ring optionally contains an oxygen atom and is optionally substituted by one or two substituents independently selected from F and OH;
R¹² and R¹³, together with the atoms to which they are attached may be linked to form an optionally unsaturated 5-7 membered heterocyclyl ring, wherein the 5- to 7-membered heterocyclyl ring optionally contains an oxygen atom and is optionally substituted by one or two substituents independently selected from F and OH;
n is 1 or 2;
and when R² and R⁹ are C₍₁₋₃₎alkyl, they are optionally substituted by fluorine.

2. A compound of formula (I) or a pharmaceutically acceptable salt thereof, wherein:
X is CH or N;
R¹ is OR³;
R³ is isopropyl;
R² is chloro or cyano;
A is (a) or (b);
R⁹ is hydrogen or methyl; and
n is 2.

3. A compound according to claim 1 selected from the group consisting of:
2-(5-{3-Chloro-4-[(1-methylethyl)oxy]phenyl}-1,3,4-thiadiazol-2-yl)-3-methyl-4,5,6,7-tetrahydro-2H-pyrazolo[4,3-c]pyridine;
3-[2-(5-{3-Chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanoic acid;
2-[(1-Methylethyl)oxy]-5-[5-(3-methyl-4,5,6,7-tetrahydro-2H-pyrazolo[4,3-c]pyridin-2-yl)-1,3,4-thiadiazol-2-yl]benzonitrile;
3-[2-(5-{3-Cyano-4-[(1-methylethyl)oxy]phenyl}-1,3,4-thiadiazol-2-yl)-3-methyl-2,4,6,7-tetrahydro-5H-pyrazolo[4,3-d]pyridin-5-yl]propanoic acid;
3-[2-(5-{3-Cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanamide;
2-(5-{3-Chloro-4-[(1-methylethyl)oxy]phenyl}-1,3,4-thiadiazol-2-yl)-4,5,6,7-tetrahydro-2H-pyrazolo[4,3-c]pyridine;
4-[2-(5-{3-Chloro-4-[(1-methylethyl)oxy]phenyl}-1,3,4-thiadiazol-2-yl)-2,4,6,7-tetrahydro-5H-pyrazolo[4,3-c]pyridin-5-yl]butanoic acid;
3-[2-(5-{3-Chloro-4-[(1-methylethyl)oxy]phenyl}-1,3,4-thiadiazol-2-yl)-2,4,6,7-tetrahydro-5H-pyrazolo[4,3-c]pyridin-5-yl]propanoic acid;
3-[2-(5-{3-Chloro-4-[(1-methylethyl)oxy]phenyl}-1,3,4-thiadiazol-2-yl)-2,4,6,7-tetrahydro-5H-pyrazolo[4,3-c]pyridin-5-yl]-1-propanol;
2-(5-{3-Chloro-4-[(1-methylethyl)oxy]phenyl}-1,3,4-thiadiazol-2-yl)-5-[2-(methylsulfonyl)ethyl]-4,5,6,7-tetrahydro-2H-pyrazolo[4,3-c]pyridine;
2-[(1-Methylethyl)oxy]-5-[5-(4,5,6,7-tetrahydro-2H-pyrazolo[4,3-c]pyridin-2-yl)-1,3,4-thiadiazol-2-yl]benzonitrile;
3-[2-(5-{3-Cyano-4-[(1-methylethyl)oxy]phenyl}-1,3,4-thiadiazol-2-yl)-2,4,6,7-tetrahydro-5H-pyrazolo[4,3-d]pyridin-5-yl]propanoic acid;
4-[2-(5-{3-Cyano-4-[(1-methylethyl)oxy]phenyl}-1,3,4-thiadiazol-2-yl)-2,4,6,7-tetrahydro-5H-pyrazolo[4,3-c]pyridin-5-yl]butanoic acid;
1-(5-{3-Chloro-4-[(1-methylethyl)oxy]phenyl}-1,3,4-thiadiazol-2-yl)-4,5,6,7-tetrahydro-1H-pyrazolo[4,3-c]pyridine;
3-[1-(5-{3-Chloro-4-[(1-methylethyl)oxy]phenyl}-1,3,4-thiadiazol-2-yl)-1,4,6,7-tetrahydro-5H-pyrazolo[4,3-c]pyridin-5-yl]propanoic acid;
4-[2-(5-{3-Chloro-4-[(1-methylethyl)oxy]phenyl}-1,3,4-thiadiazol-2-yl)-4,5,7,8-tetrahydropyrazolo[3,4-d]azepin-6(2H)-yl]butanoic acid;
2-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-N-[2-hydroxy-1-(hydroxymethyl)ethyl]acetamide;
2-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-N-[(1S)-2-hydroxy-1-methylethyl]acetamide;
2-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-N-[(1R)-2-hydroxy-1-methylethyl]acetamide;
2-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-N-(2-hydroxyethyl)acetamide;
2-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-N-[(2S)-2-hydroxypropyl]acetamide;
2-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-N-[(2S)-2-hydroxypropyl]acetamide;
2-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-N-[(2R)-2-hydroxypropyl]acetamide;
5-{5-[5-(2-hydroxyethyl)-3-methyl-4,5,6,7-tetrahydro-2H-pyrazolo[4,3-c]pyridin-2-yl]-1,3,4-thiadiazol-2-yl}-2-[(1-methylethyl)oxy]benzonitrile;
5-(5-{5-[2-hydroxy-1-(hydroxymethyl)ethyl]-3-methyl-4,5,6,7-tetrahydro-2H-pyrazolo[4,3-c]pyridin-2-yl}-1,3,4-thiadiazol-2-yl)-2-[(1-methylethyl)oxy]benzonitrile;
5-(5-{5-[(2S)-2,3-dihydroxypropyl]-3-methyl-4,5,6,7-tetrahydro-2H-pyrazolo[4,3-c]pyridin-2-yl}-1,3,4-thiadiazol-2-yl)-2-[(1-methylethyl)oxy]benzonitrile;
5-(5-{5-[(2R)-2,3-dihydroxypropyl]-3-methyl-4,5,6,7-tetrahydro-2H-pyrazolo[4,3-c]pyridin-2-yl}-1,3,4-thiadiazol-2-yl)-2-[(1-methylethyl)oxy]benzonitrile;
5-{5-[5-(3-hydroxypropyl)-3-methyl-4,5,6,7-tetrahydro-2H-pyrazolo[4,3-c]pyridin-2-yl]-1,3,4-thiadiazol-2-yl}-2-[(1-methylethyl)oxy]benzonitrile;
5-[5-(5-glycyl-3-methyl-4,5,6,7-tetrahydro-2H-pyrazolo[4,3-c]pyridin-2-yl)-1,3,4-thiadiazol-2-yl]-2-[(1-methylethyl)oxy]benzonitrile;
5-[5-(5-{N-[(1R)-2-hydroxy-1-methylethyl]glycyl}-3-methyl-4,5,6,7-tetrahydro-2H-pyrazolo[4,3-c]pyridin-2-yl)-1,3,4-thiadiazol-2-yl]-2-[(1-methylethyl)oxy]benzonitrile;
5-[5-(5-{N-[(1S)-2-hydroxy-1-methylethyl]glycyl}-3-methyl-4,5,6,7-tetrahydro-2H-pyrazolo[4,3-c]pyridin-2-yl)-1,3,4-thiadiazol-2-yl]-2-[(1-methylethyl)oxy]benzonitrile;
5-[5-(5-{N-[(2R)-2-hydroxypropyl]glycyl}-3-methyl-4,5,6,7-tetrahydro-2H-pyrazolo[4,3-c]pyridin-2-yl)-1,3,4-thiadiazol-2-yl]-2-[(1-methylethyl)oxy]benzonitrile;
5-{5-(5-[N-[(2S)-2-hydroxypropyl]glycyl}-3-methyl-4,5,6,7-tetrahydro-2H-pyrazolo[4,3-c]pyridin-2-yl)-1,3,4-thiadiazol-2-yl]-2-[(1-methylethyl)oxy]benzonitrile;
5-[5-(5-{N-[(2S)-2,3-dihydroxypropyl]glycyl}-3-methyl-4,5,6,7-tetrahydro-2H-pyrazolo[4,3-c]pyridin-2-yl)-1,3,4-thiadiazol-2-yl]-2-[(1-methylethyl)oxy]benzonitrile;
5-(5-{5-[N-(2-hydroxyethyl)glycyl}-3-methyl-4,5,6,7-tetrahydro-2H-pyrazolo[4,3-c]pyridin-2-yl]-1,3,4-thiadiazol-2-yl)-2-[(1-methylethyl)oxy]benzonitrile;
5-[5-(5-{N-[2-hydroxy-1-(hydroxymethyl)ethyl]glycyl}-3-methyl-4,5,6,7-tetrahydro-2H-pyrazolo[4,3-c]pyridin-2-yl)-1,3,4-thiadiazol-2-yl]-2-[(1-methylethyl)oxy]benzonitrile
5-[5-(5-{N-[(2R)-2,3-dihydroxypropyl]glycyl}-3-methyl-4,5,6,7-tetrahydro-2H-pyrazolo[4,3-c]pyridin-2-yl)-1,3,4-thiadiazol-2-yl]-2-[(1-methylethyl)oxy]benzonitrile;
[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]acetic acid;
2-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-1,3-propanediol;
(2R)-3-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-1,2-propanediol;
methyl (2R)-3-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-2-hydroxypropanoate;
(2S)-3-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-1,2-propanediol;
2-[(1-methylethyl)oxy]-5-[5-(3-methyl-4,5,6,7-tetrahydro-2H-pyrazolo[3,4-c]pyridin-2-yl)-1,3,4-thiadiazol-2-yl]benzonitrile; and
2-[2-(5-{3-cyano-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-N-(2-hydroxy-1,1-dimethylethyl)acetamide,
or a salt or ester thereof.

4. A compound according to claim 1, which is 3-[2-(5-{3-Chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]propanoic acid or a salt or ester thereof.

5. A compound according to claim 1, which is 2-[2-(5-{3-chloro-4-[(1-methylethyl)oxy]phenyl}-1,3,4-thiadiazol-2-yl)-3-methyl-2,4,6,7-tetrahydro-5H-pyrazolo[4,3-c]pyridin-5-yl]-1,3-propanediol or a salt or ester thereof.

6. A method of treating a condition or disorder mediated by S1P1 comprising administering to a subject with said condition or disorder a therapeutically effective amount of a compound of formula (I) or a pharmaceutically acceptable salt thereof according to claim 1.

7. A method according to claim 6, wherein the condition or disorder is multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

8. A method according to claim 7, wherein the condition is psoriasis.

9. (canceled)

10. (canceled)

11. (canceled)

12. A pharmaceutical composition comprising a compound of formula (a) or a pharmaceutically acceptable salt thereof according to claim 1.

13. (canceled)

14. (canceled)
